(19) United States
(12) Patent Application Publication
(10) Pub. No.: US 2008/0234271 A1
(43) Pub. Date: Sep. 25, 2008

(54) Arylphenylamino- and Arylphenylether-Sulfide Derivatives, Useful For the Treatment of Inflammatory and Immune Diseases, and Pharmaceutical Compositions Containing Them

(75) Inventors: Kevin Guckian, Marlborough, MA (US); Daniel Scott, Weston, MA (US); Irina Jacobson, Sammamish, WA (US); C.Gregory Sowell, Mukilteo, WA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: ICOS Corporation, Bothell, WA (US); Biogen Idec MA Inc., Cambridge, MA (US)

(21) Appl. No.: 11/579,098

(22) PCT Filed: Apr. 28, 2005
(86) PCT No.: PCT/US2005/014802

§ 371 Date: Mar. 14, 2008

Related U.S. Application Data
(60) Provisional application No. 60/565,838, filed on Apr. 28, 2004.
(60) Provisional application No. 60/620,277, filed on Oct. 20, 2004.

(51) Int. Cl. A61K 31/5377; C07D 413/06; A61P 29/00

(52) U.S. Cl. 514/235.5; 544/152; 514/231.5; 544/131

(57) ABSTRACT

The present invention relates in part to compounds of formulas (I) and (III): and pharmaceutically-acceptable salts and prodrugs thereof. These compounds can be useful for treating diseases such as inflammatory and immune diseases. The present invention also relates to pharmaceutical compositions comprising these compounds, and to methods of inhibiting inflammation or suppressing immune response in a subject.

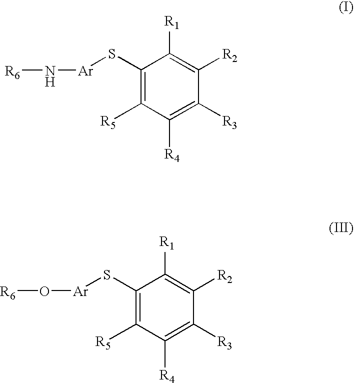

### CROSS-REFERENCE TO RELATED APPLICATIONS

This application claims the benefit of U.S. provisional application Ser. No. 60/565,838, filed Apr. 28, 2004, and U.S. provisional application Ser. No. 60/620,277, filed Oct. 20, 2004, the contents of which are incorporated herein by reference in their entirety.

### FIELD OF THE INVENTION

The present invention relates to small molecule LFA-1 antagonists that are useful for treating inflammatory and immune diseases, to pharmaceutical compositions comprising these compounds, to methods of making these compounds, and to methods of inhibiting inflammation, or modulating or suppressing an immune response in a mammal.

### BACKGROUND OF THE INVENTION

Leukocyte function-associated antigen-I (referred to herein as “LFA-1” and alternatively known as CD11a/CD18) is a heterodimeric cell surface adhesion receptor expressed on all leukocytes. The known counter-receptors for LFA-1 are intracellular adhesion molecules-1, 2, and 3 (ICAM-1, ICAM-2, and ICAM-3). The functional interaction of LFA-1/ICAMs is often associated with a number of inflammatory processes. LFA-1 can serve a dual role in inflammatory responses: it can function as a co-stimulatory molecule during the activation of T cells and can participate in the adhesive interactions associated with the recirculation of leukocytes (for review see; T. A. Springer et al., Nature 1990, 346, 425-434 and M. Lub et al., Immunology Today 1995, 16, 479483).

Activated T cells are often key mediators in an immune response, functioning either through the secretion of cytokines and chemokines that draw other immune cells to the site of inflammation or through the acquisition of effector functions. The signaling events that lead to T cell activation can arise as a result of the adhesive interaction between T cells and antigen presenting cells (APCs). T cells express specific T cell receptors (TCRs) that recognize their unique cognate antigen as part of an antigen/MHC (major histocompatibility complex) complex on the surface of APCs. The avidity of the TCR interaction is weak and additional adhesive interactions like those conferred by LFA-1/ICAM-1 may be required to stabilize the cell-cell contact and provide co-stimulatory signals. Within the contact site, antigen receptors, adhesion molecules and co-stimulatory molecules are coordinated in a spatio-temporal manner to form a stable “immunological synapse” (IS) that is required for achieving T cell activation. See Monks et al., Nature 395(6697):82-86, 1998; S.-Y.Tseng et al., Curr Opin Cell Biol 14(5):575-580,2002; M. Krummel et al., Curr Opin Immunol 14(1):66-74, 2002. It is also known that inhibition of LFA-1/ICAM-1 interaction with LFA-1 specific blocking antibodies prevents T cell activation in vitro (Calhoun et al., Transplantation 68:1144, 1999) and in numerous animal models of inflammation.

Inflammation typically results from a cascade of events that includes vasodilation accompanied by increased vascular permeability and exudation of fluid and plasma proteins. This disruption of vascular integrity precedes or coincides with an infiltration of inflammatory cells. Inflammatory mediators generated at the site of the initial lesion serve to recruit inflammatory cells to the site of injury. These mediators (chemokines such as IL-8, MCP-1, MIP-1, and RANTES, complement fragments and lipid mediators) have chemotactic activity for leukocytes and attract the inflammatory cells to the inflamed lesion. These chemotactic mediators, which cause circulating leukocytes to localize at the site of inflammation, require the cells to cross the vascular endothelium at a precise location. This leukocyte recruitment is accomplished by a process called cell adhesion.

Cell adhesion occurs through a coordinately regulated series of steps that allow the leukocytes to first adhere to a specific region of the vascular endothelium and then cross the endothelial barrier to migrate to the inflamed tissue (T. A. Springer, Cell, 76:301-314,1994; M. B. Lawrence et al., Cell, 65:859-873,1991; U. von Adrian et al., Proc. Natl. Acad. Sci. USA, 88:7538-7542, 1991; and K. Ley et al., Blood, 77:2553-2555, 1991). These steps are mediated by families of adhesion molecules such as integrins, Ig supergene family members, and selectins, which are expressed on the surface of the circulating leukocytes and on the vascular endothelial cells.

Initially, leukocytes roll along the vascular endothelial cell lining in the region of inflammation. The rolling step may be mediated by either selectin-carbohydrate interactions or integrin-Ig superfamily member interactions between the leukocyte and the luminal surface of inflamed endothelium. The endothelial expression of both selectins and Ig superfamily members are up-regulated in response to the action of inflammatory mediators such as TNF-α and interleukin-1. Rolling decreases the velocity of the circulating leukocyte in the region of inflammation and allows the cells to more firmly adhere to the endothelial cell. The firm adhesion is accomplished by the interaction of integrin molecules that are present on the surface of the rolling leukocytes and their counter-receptors (the Ig superfamily molecules) on the surface of the endothelial cell. The Ig superfamily molecules or cell adhesion molecules (CAMs) are either not expressed or are expressed at low levels on normal vascular endothelial cells. The adhesion process relies on the induced expression of selectins and CAMs on the surface of vascular endothelial cells to mediate the rolling and firm adhesion of leukocytes to the vascular endothelium. The final event in the adhesion process is the extravasation of leukocytes through the endothelial cell barrier and their migration along a chemotactic gradient to the site of inflammation.

The interaction of ICAM-1 (CD54) on endothelial cells with the integrin LFA-1 on leukocytes plays an important role in endothelial-leukocyte contact. Leukocytes bearing high-affinity LFA-1 adhere to endothelial cells through interaction with ICAM-1, initiating the process of extravasation from the vasculature into the surrounding tissues. Thus, an agent that blocks the ICAM-1/LFA-1 interaction suppresses these early steps in the inflammatory response. Consistent with this background, ICAM-1 knockout mice have numerous abnormalities in their inflammatory responses.

Compounds that bind to the inserted-domain (I-domain) of LFA-1, can interrupt endothelial cell-leukocyte adhesion by blocking the interaction of LFA-1 with ICAM-1 and ICAM-3. These compounds can be useful for the treatment or prophylaxis of diseases in which leukocyte trafficking or T-cell activation plays a role, such as acute and chronic inflammatory diseases, autoimmune diseases, tumor metastasis, allograft rejection, and reperfusion injury.

### SUMMARY OF THE INVENTION

The present invention relates to novel compounds and pharmaceutical compositions comprising these compounds. The compounds of the invention can bind to the I-domain of LFA-1.

In one embodiment, the compounds of this invention are diaromatic sulfides, such as diaryl sulfides or aryl-heteroaryl sulfides, that are substituted with a cinnamide group. The cinnamide functionality may be placed either ortho- or para- to the linking sulfur atom. Appropriate substitution of either or both aromatic rings can be used to modulate a variety of biochemical, physicochemical and pharmacokinetic properties. The cinnamide group can be readily modified; a variety of secondary and tertiary amides can be active, and alternatively a heterocyclic ring may be attached at this position. Modifications of this cinnamide functionality can be useful in modulating physicochemical and pharmacokinetic properties.

In one embodiment, the compounds of the invention are diaryl sulfides and aryl-heteroaryl sulfides that are substituted with a cinnamide group at one aryl, and a secondary amine at the other aryl or heteroaryl. The invention further relates to methods of making diaryl sulfides and aryl-heteroaryl sulfides.

The compounds of the invention can be used to treat diseases such as acute and chronic inflammatory diseases, autoimmune diseases, tumor metastasis, allograft rejection, and reperfusion injury. Thus, certain embodiments of the invention include methods of treating inflammatory and immune diseases, and methods of inhibiting inflammation or suppressing immune response in a mammal.

It is to be understood that both the foregoing general description and the following detailed description are exemplary and explanatory only and are not restrictive of the invention, as claimed.

### DETAILED DESCRIPTION

Unless otherwise specified, the chemical groups identified below refer to both unsubstituted and substituted groups.

The term “aldehyde” as used herein refers to the radical —CHO.

The term “aldehyde hydrazone” as used herein refers to the radical —CH═N—NR₁₂R₁₃, where R₁₂ and R₁₃, are independently selected from hydrogen, alkyl, aryl, or cycloalkyl.

The term “alkanoyl” as used herein refers to a carbonyl group attached to an alkyl group.

The term “alkanoylamino” as used herein refers to an alkanoyl group attached to an amino group, e.g., —C(O)-alkyl-amino-.

The term “alkanoylaminoalkyl” as used herein refers to an alkanoylamino group attached to an alkyl group, e.g., —C(O)-alkyl-amino-alkyl-.

The term “alkanoyloxy” as used herein refers to an alkanoyl group attached to an oxygen, e.g., —C(O)-alkyl-O—.

The term “alkanoyloxyalkyl” as used herein refers to an alkanoyloxy group attached to an alkyl group, e.g., —C(O)-alkyl-O-alkyl-.

The term “alkenoxycarbonyl” as used herein refers to an alkenoxy group attached to a carbonyl group, e.g., —O-alkene-C(O)—.

The term “alkenyl” as used herein refers to an unsaturated straight or branched chain of 2-20 carbon atoms having at least one carbon-carbon double bond, such as a straight or branched chain group of 2-12, 2-10, or 2-6 carbon atoms.

The term “alkoxy” as used herein refers to an alkyl group attached to an oxygen. “Alkoxy” groups can optionally contain alkenyl (“alkenoxy”) or alkynyl (“alkynoxy”) groups.

The term “alkoxyalkanoyl” as used herein refers to an alkoxy group attached to an alkanoyl group, e.g., -alkyl-O-C(O)-alkyl-.

The term “alkoxyalkoxy” as used herein refers to an alkoxy group attached to another alkoxy group, e.g., —O-alkyl-O-alkyl-.

The term “alkoxyalkyl” as used herein refers to an alkoxy group attached to an alkyl group, e.g., -alkyl-O-alkyl-.

The term “alkoxyalkylcarbonyl” as used herein refers to an alkoxyalkyl group attached to a carbonyl group, e.g., -alkyl-O-alkyl-C(O)—.

The term “alkoxycarbonyl” as used herein refers to an alkoxy group attached to a carbonyl group, e.g., —C(O)-O-alkyl-.

The term “alkoxycarbonylalkyl” as used herein refers to an alkoxycarbonyl group attached to an alkyl group, e.g., -alkyl-C(O)—O-alkyl-.

The term “alkoxycarbonylamido” as used herein refers to an alkoxycarbonyl group attached to an amido group, e.g., -amido-C(O)—O-alkyl-.

The term “alkyl” as used herein refers to a saturated straight or branched chain group of 1-20 carbon atoms, such as a straight or branched chain group of 1-12, 1-10, or 1-6 carbon atoms.

The term “alkyl(alkoxycarbonylalkyl) amino” as used herein refers to an amino group substituted with one alkyl group and one alkoxycarbonylalkyl group, e.g., -alkyl-C(O)—O-alkyl-amino-alkyl-.

The term “alkylsulfonyl” as used herein refers to an alkyl group attached to a sulfonyl group. “Alkylsulfonyl” groups can optionally contain alkenyl or alkynyl groups.

The term “alkylsulfonylamido” as used herein refers to an alkylsulfonyl group attached to an amido group, e.g., -alkyl-SO₂-amido-.

The term “alkylthio” as used herein refers to an alkyl group attached to a sulfur atom. “Alkylthio” groups can optionally contain alkenyl or alkynyl groups.

The term “alkynyl” as used herein refers to an unsaturated straight or branched chain group of 2-20 carbon atoms having at least one carbon-carbon triple bond, such as a straight or branched chain group of 2-12, 2-10, or 2-6 carbon atoms.

The term “amido” as used herein refers to a radical of the form —R₁₆C(O)N(R₁₄)—, —R₁₆C(O)N(R₁₄)R₁₅—, or —C(O)NR₁₄R₁₅, where R₁₄ and R₁₅ are each independently selected from hydrogen, alkyl, alkanoyl, alkenyl, alkoxy, alkynyl, aryl, carboxy, cycloalkyl, ester, ether, heterocyclyl, hydroxy, ketone, thio, and sulfonyl, and R₁₆ is selected from hydrogen, alkyl, alkoxy, amido, amino, aryl, cycloalkyl, ester, ether, heterocyclyl, halogen, hydroxy, ketone, and thio. The amido can be attached to another group through the carbon, the nitrogen, R₁₄, R₁₅, or R₁₆. The amido also may be cyclic, for example R₁₄ and R₁₅, R₁₆ and R₁₄, or R₁₆ and R₁₅ may be joined to form a 3- to 12-membered ring, such as a 3- to 10-membered ring. The term “amido” encompasses groups such as alkanoylaminoalkyl, amidoalkyl (attached to the parent molecular group through the alkyl), alkylamido (attached to the parent molecular group through the amido), arylamido, amidoaryl, sulfonamide, etc. The term “amido” also encompasses groups such as urea, carbamate, and cyclic versions thereof.

The term “amidoalkoxy” as used herein refers to an amido group attached to an alkoxy group, e.g., -amido-alkyl-O—.

The term “amino” as used herein refers to a radical of the form —NR₁₇R₁₈, —N(R₁₇)R₁₈—, or —R₁₈N(R₁₇)R₁₉— where R₁₇, R₁₈, and R₁₉ are independently selected from hydrogen, alkyl, alkenyl, alkanoyl, alkoxy, alkynyl, amido, amino, aryl, carboxy, cycloalkyl, ester, ether, heterocyclyl, hydroxy, ketone, thio, and sulfonyl. The amino can be attached to the parent molecular group through the nitrogen, R₁₇, R₁₈ or R₁₉. The amino also may be cyclic, for example any two of R₁₇, R₁₈, and R₁₉ may be joined together or with the N to form a 3- to 12-membered ring, e.g., morpholino or piperidinyl. The term “amino” encompasses groups such as aminoalkyl (attached to the parent molecular group through the alkyl), alkylamino (attached to the parent molecular group through the amino), arylamino, aminoaryl, sulfonamino, etc. The term amino also includes the corresponding quaternary ammonium salt of any amino group, e.g., —[N(R₁₇)(R₁₈)(R₁₉)]⁺.

The term “aminoalkanoyl” as used herein refers to an amino group attached to an alkanoyl group, e.g., —C(O)-alkyl-amino-.

The term “aminoalkoxy” as used herein refers to an amino group attached to an alkoxy group, e.g., —O-alkyl-amino-.

The term “aminocarbonyl” as used herein refers to an amino group attached to a carbonyl group.

The term “aminosulfonyl” as used herein refers to an amino group attached to an sulfonyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be optionally substituted with groups selected from alkyl, aldehyde, alkanoyl, alkoxy, amino, amido, aryl, carboxy, cyano, cycloalkyl, ester, ether, halogen, heterocyclyl, hydroxy, ketone, nitro, sulfonate, sulfonyl, and thio.

The term “arylalkanoyl” as used herein refers to an aryl group attached to an alkanoyl group, e.g., —C(O)-alkyl-aryl- or -alkyl-C(O)-aryl-.

The term “arylalkoxy” as used herein refers to an aryl group attached to an alkoxy group, e.g., —O-alkyl-aryl- or -aryl-O-alkyl-.

The term “arylalkoxycarbonyl” as used herein refers to an arylalkoxy group attached to a carbonyl group.

The term “arylalkyl” as used herein refers to an aryl group attached to an alkyl group.

The term “arylalkylamido” as used herein refers to an arylalkyl group attached to an amido group, e.g., -alkyl-aryl-amido- or -aryl-alkyl-amido-.

The term “arylalkylsulfonyl” as used herein refers to an arylalkyl group attached to an sulfonyl group, e.g., -alkyl-aryl-sulfonyl- or -aryl-alkyl-sulfonyl-.

The term “arylcarboxy” as used herein refers to an aryl group attached to a carboxy group, e.g., -aryl-COOH or salts such as -aryl-COONa.

The term “arylcarboxyamido” as used herein refers to an arylcarboxy group attached to an amido group, e.g., -amido-aryl-COOH or salts such as -amido-aryl-COONa.

The term “aryloxy” as used herein refers to an aryl group attached to an oxygen atom.

The term “aryloxycarbonyl” as used herein refers to an aryloxy group attached to a carbonyl group, e.g., —C(O)—O-aryl- or —O-aryl-C(O)—.

The term “arylsulfonyl” as used herein refers to an aryl group attached to a sulfonyl group, e.g., —S(O)₂-aryl-.

The term “arylsulfonylamido” as used herein refers to an arylsulfonyl group attached to an amido group, e.g., -amido-S(O)₂-aryl-.

The term “carbonyl” as used herein refers to the radical —C(O)—.

The term “carbonyl-containing group” as used herein refers to any group containing the radical —C(O)—.

The term “carboxy” as used herein refers to the radical —COOH. The term “carboxy” also includes salts such as —COONa, etc.

The term “carboxyalkoxy” as used herein refers to an alkoxy group attached to a carboxy group, e.g., —O-alkyl-COOH or salts such as —O-alkyl-COONa, etc.

The term “carboxyalkyl” as used herein refers to a carboxy group attached to an alkyl group, e.g., -alkyl-COOH or salts such as -alkyl-COONa, etc. “Carboxylalkyls” can optionally contain alkenyl or alkynyl groups.

The term “carboxyalkylcarbonyl” as used herein refers to a carboxyalkyl group attached to a carbonyl group, e.g., —C(O)-alkyl-COOH or salts such as —C(O)-alkyl-COONa, etc.

The term “carboxyalkylcycloalkyl” as used herein refers to a carboxyalkyl group attached to a cycloalkyl group, e.g., -cycloalkyl-alkyl-COOH or salts such as -cycloalkyl-alkyl-COONa, etc.

The term “carboxyamido” as used herein refers to an amido group attached to a carboxy group, e.g., -amido-COOH or salts such as -amido-COONa, etc.

The term “carboxyamino” as used herein refers to an amino group attached to a carboxy group, e.g., -amino-COOH or salts such as -amino-COONa, etc.

The term “carboxyaminocarbonyl” as used herein refers to a carboxyamino group attached to a carbonyl group, e.g., —C(O)-amino-COOH or salts such as —C(O)-amino-COONa, etc.

The term “carboxycarbonyl” as used herein refers to a carboxy group attached to a carbonyl group, e.g., —C(O)-COOH or salts such as —C(O)—COONa, etc.

The term “carboxycycloalkoxy” as used herein refers to a cycloalkoxy group attached to a carboxy group, e.g., —O-cycloalkyl-COOH or salts such as —C(O)-cycloalkyl —COONa, etc.

The term “carboxycycloalkyl” as used herein refers to a cycloalkyl group attached to a carboxy group, e.g., -cycloalkyl-COOH or salts such as -cycloalkyl —COONa, etc.

The term “carboxycycloalkylalkyl” as used herein refers to a carboxycycloalkyl group attached to an alkyl group, e.g., -alkyl-cycloalkyl-COOH or salts such as -alkyl-cycloalkyl-COONa, etc.

The term “carboxythioalkoxy” as used herein refers to a thioalkoxy group attached to a carboxy group, e.g., —S-alkyl-COOH or salts such as —S-alkyl-COONa, etc.

The term “cyano” as used herein refers to the radical —CN.

The term “cycloalkoxy” as used herein refers to a cycloalkyl group attached to an oxygen, e.g., —O-cycloalkyl-.

The term “cycloalkyl” as used herein refers to a monovalent saturated or unsaturated cyclic, bicyclic, or bridged bicyclic hydrocarbon group of 3-12 carbons derived from a cycloalkane by the removal of a single hydrogen atom, e.g., cyclohexanes, cyclohexenes, cyclopentanes, and cyclopentenes. Cycloalkyl groups may be optionally substituted with alkyl, alkylthio, aldehyde, alkanoyl, alkoxy, amido, amino, aminothiocarbonyl, aryl, arylcarbonyl, arylthio, carboxy, carboxyalkyl, cyano, cycloalkyl, ester, ether, halogen, heterocyclyl, heterocyclylcarbonyl, hydroxy, ketone, nitro, sulfonate, sulfonyl, and thiol. Cycloalkyl groups can be optionally bonded to the parent molecular group through any of its substituents. Cycloalkyl groups can be optionally fused to other cycloalkyl, aryl, or heterocyclyl groups.

The term “cycloalkylalkyl” as used herein refers to a cycloalkyl group attached to an alkyl group, e.g., -alkyl-cycloalkyl-.

The term “ester” refers to a radical having the structure —C(O)O—, —C(O)O—R₂₀—, —R₂₁C(O)O—R₂₀—, or —R₂₁C(O)O—, where O is not bound to hydrogen, and R₂₀ and R₂₁ can independently be alkyl, alkenyl, alkynyl, aryl, cycloalkyl, ester, ether, heterocyclyl, ketone, and thio. R₂₁ can be a hydrogen, but R₂₀ cannot be hydrogen. The ester may be cyclic, for example the carbon atom and R₂₀, the oxygen atom and R₂₁, or R₂₀ and R₂₁ may be joined to form a 3- to 12-membered ring. Exemplary esters include alkoxyalkanoyl, alkoxycarbonyl, alkoxycarbonylalkyl, etc. Esters also include carboxylic acid anhydrides and acid halides.

The term “ether” refers to a radical having the structure —R₂₂O—R₂₃—, where R₂₂ and R₂₃ can independently be alkyl, alkenyl, alkynyl, aryl, cycloalkyl, or heterocyclyl. The ether can be attached to the parent molecular group through R₂₂ or R₂₃. Exemplary ethers include alkoxyalkyl and alkoxyaryl groups. Ether also includes polyethers, e.g., where one or both of R₂₂ and R₂₃ are ethers.

The terms “halo” or “halogen” as used herein refer to F, Cl, Br, or I.

The term “haloalkyl” as used herein refers to an alkyl group substituted with one or more halogen atoms. “Haloalkyls” can optionally contain alkenyl or alkynyl groups.

The term “heteroaryl” as used herein refers to a mono-, bi-, or multi-cyclic, aromatic ring system containing one, two, or three heteroatoms such as nitrogen, oxygen, and sulfur. Heteroaryls can be optionally substituted with one or more substituents including alkyl, alkenyl, alkynyl, aldehyde, alkoxy, amido, amino, aryl, carboxy, cyano, cycloalkyl, ester, ether, halogen, heterocyclyl, hydroxy, ketone, nitro, sulfonate, sulfonyl, and thio. Heteroaryls can also be fused to non-aromatic rings.

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be optionally substituted with one or more substituents including alkyl, alkenyl, alkynyl, aldehyde, alkylthio, alkanoyl, alkoxy, alkoxycarbonyl, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, oxo, nitro, sulfonate, sulfonyl, and thiol.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

Heterocycles also include bridged bicyclic groups where a monocyclic heterocyclic group can be bridged by an alkylene group such as

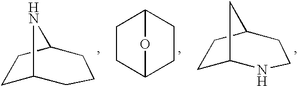

Heterocycles also include compounds of the formula

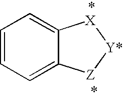

where X* and Z* are independently selected from —CH₂—, —CH₂NH—, —CH₂O—, —NH— and —O—, with the proviso that at least one of X* and Z* is not —CH₂—, and Y* is selected from —C(O)— and —(C(R″)₂)ᵥ—, where R″ is hydrogen or alkyl of one to four carbons, and v is 1-3. These heterocycles include 1,3-benzodioxolyl, 1,4-benzodioxanyl, and 1,3-benzimidazol-2-one.

The term “heterocyclylalkyl” as used herein refers to a heterocyclic group attached to an alkyl group. “Heterocyclylalkyls” can optionally contain alkenyl or alkynyl groups.

The term “heterocyclylalkylcarbonyl” as used herein refers to a heterocyclylalkyl group attached to a carbonyl, e.g., —C(O)-alkyl-heterocyclyl- or -alkyl-heterocyclyl-C(O)—.

The term “heterocyclylalkylsulfonyl” as used herein refers to a heterocyclylalkyl group attached to a sulfonyl, e.g., —SO₂-alkyl-heterocyclyl- or -alkyl-heterocyclyi-SO₂—.

The term “heterocyclylamido” as used herein refers to a heterocyclyl group attached to an amido group.

The term “heterocyclylamino” as used herein refers to a heterocyclyl group attached to an amino group.

The term “heterocyclylcarbonyl” as used herein refers to a heterocyclyl group attached to a carbonyl group.

The term “heterocyclylsulfonyl” as used herein refers to a heterocyclyl group attached to an —SO₂-group.

The term “heterocyclylsulfonylamido” as used herein refers to a heterocyclylsulfonyl group attached to an amido group.

The term “hydroxyl” as used herein refers to the radical —OH.

The term “hydroxyalkanoyl” as used herein refers to a hydroxy radical attached to an alkanoyl group, e.g., —C(O)-alkyl-OH.

The term “hydroxyalkoxy” as used herein refers to a hydroxy radical attached to an alkoxy group, e.g., —O-alkyl-OH.

The term “hydroxyalkoxyalkyl” as used herein refers to a hydroxyalkoxy group attached to an alkyl group, e.g., -alkyl-O-alkyl-OH.

The term “hydroxyalkyl” as used herein refers to a hydroxy radical attached to an alkyl group.

The term “hydroxyalkylamido” as used herein refers to a hydroxyalkyl group attached to an amido group, e.g., -amido-alkyl-OH.

The term “hydroxyamido” as used herein refers to an amido group attached to a hydroxy radical.

The term “hydroxyamino” as used herein refers to an amino group attached to a hydroxy radical.

The term “ketone” refers to a radical having the structure —R₂₄—C(O)—R₂₅—. The ketone can be attached to another group through R₂₄ or R₂₅. R₂₄ or R₂₅ can be alkyl, alkenyl, alkynyl, cycloalkyl, heterocyclyl or aryl, or R₂₄ or R₂₅ can be joined to form a 3- to 12-membered ring. Exemplary ketones include alkanoylalkyl, alkylalkanoyl, etc.

The term “nitro” as used herein refers to the radical —NO₂.

The term “oxo” as used herein refers to an oxygen atom with a double bond to another atom. For example, a carbonyl is a carbon atom with an oxo group.

The term “perfluoroalkyl” as used herein refers to an alkyl group in which all of the hydrogen atoms have been replaced by fluorine atoms.

The term “phenyl” as used herein refers to a monocyclic carbocyclic ring system having one aromatic ring. The phenyl group can also be fused to a cyclohexane or cyclopentane ring. The phenyl groups of this invention can be optionally substituted with one or more substituents including alkyl, alkenyl, alkynyl, aldehyde, alkoxy, amido, amino, aryl, carboxy, cyano, cycloalkyl, ester, ether, halogen, heterocyclyl, hydroxy, ketone, nitro, sulfonate, sulfonyl, and thio.

The term “sulfonamido” or “sulfonamide” as used herein refers to a radical having the structure —(R₂₇)—N—S(O)₂—R₂₈— or —R₂₆(R₂₇)—N—S(O)₂—R₂₈, where R₂₆, R₂₇, and R₂₈ can be, for example, hydrogen, alkyl, alkenyl, alkynyl, aryl, cycloalkyl, and heterocyclyl. Exemplary sulfonamides include alkylsulfonamides (e.g., where R₂₈ is alkyl), arylsulfonamides (e.g.,.where R₂₈ is aryl), cycloalkyl sulfonamides (e.g., where R₂₈ is cycloalkyl), heterocyclyl sulfonamides (e.g., where R₂₈ is heterocyclyl), etc.

The term “sulfonate” as used herein refers to the radical —SO₃H. Sulfonate also includes salts such as SO₃Na, etc.

The term “sulfonyl” as used herein refers to a radical having the structure R₂₉SO₂—, where R₂₉ can be alkyl, alkenyl, alkynyl, amino, amido, aryl, cycloalkyl, and heterocyclyl, e.g., alkylsulfonyl.

The term “sulfonylalkylamido” as used herein refers to an alkylamido group attached to a sulfonyl group, e.g. -amido-alkyl-SO₂—.

The term “sulfonylalkylsulfonyl” as used herein refers to an alkylsulfonyl group attached to a sulfonyl group, e.g., —SO₂-alkyl-SO₂—.

The term “thio” as used herein refers to radical having the structure R₃₀S—, where R₃₀ can be hydrogen, alkyl, aryl, cycloalkyl, heterocyclyl, amino, and amido, e.g., alkylthio, arylthio, thiol, etc. “Thio” can also refer to a radical where the oxygen is replaced by a sulfur, e.g., —N-C(S)— is thioamide or aminothiocarbonyl, alkyl-S— is thioalkoxy (synonymous with alkylthio).

“Alkyl,” “alkenyl,” and “alkynyl” groups, collectively referred to as “saturated and unsaturated hydrocarbons,” can be optionally substituted with or interrupted by at least one group selected from aldehyde, alkoxy, amido, amino, aryl, carboxy, cyano, cycloalkyl, ester, ether, halogen, heterocyclyl, hydroxy, ketone, nitro, sulfonate, sulfonyl, thio, O, S, and N.

The term “pharmaceutically-acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the invention.

The term “prodrug,” as used herein, represents compounds that are rapidly transformed in vivo to the parent compound of the formulas described herein, for example, by hydrolysis in blood. A discussion is provided in T. Higuchi and V. Stella, Pro-drugs as Novel Delivery Systems, Vol. 14 of the ACS Symposium Series, and in Edward B. Roche, ed., Bioreversible Carriers in Drug Design, American Pharmaceutical Association and Pergamon Press, 1987, both of which are incorporated herein by reference.

Compounds of the present invention can exist as stereoisomers when asymmetric or stereogenic centers are present. These compounds are designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers are designated “(±)”. Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

One embodiment of the present invention provides a compound of formula I:

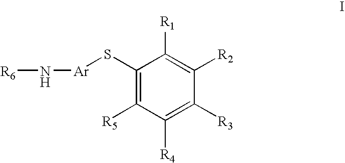

and pharmaceutically-acceptable salts and prodrugs thereof,

wherein R₁, R₂, R₃, R₄, and R₅, are independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

alternatively, R₁, R₂, R₃, R₄, R₅, and R₆ may independently be aminothiocarbonyl,

R₆ is selected from unsubstituted alkyls, unsubstituted saturated cycloalkyls, unsubstituted carboxyalkyls, and unsubstituted heterocyclylalkyls,

alternatively, R₆ may be a unsubstituted saturated carboxycycloalkyl,

wherein the unsubstituted saturated cycloalkyls, unsubstituted saturated carboxycycloalkyls, unsubstituted carboxyalkyls, and unsubstituted heterocyclylalkyls are bonded to the NH of formula I through the alkyl group,

with the proviso that the heterocyclylalkyl is not

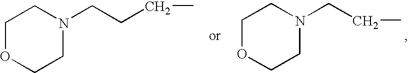

with the proviso that at least one of R₁ or R₃ is cis-cinnamide or trans-cinnamide defined as

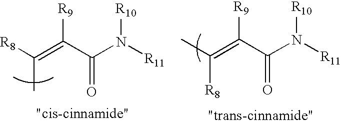

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, sulfonate, sulfonyl, thio, and other carbonyl-containing groups,

wherein R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups,

R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and

wherein R₁ and R₂, and R₄ and R₅ can be joined to form a 5- to 7-membered cycloalkyl or heterocyclyl ring when R₃ is the cinnamide, and R₂ and R₃, R₃ and R₄, and R₄ and R₅ can be joined to form a 5- to 7- membered ring when R₁ is the cinnamide,

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups.

In one embodiment, R₆ is selected from:

C₁₋₆ unsubstituted alkyls, such as methyl, ethyl, and propyl;

C₂₋₇ unsubstituted carboxyalkyls, such as —CH(CH₃)—CH₂—CH₂—C(O)—OH;

C₃₋₇ unsubstituted saturated cycloalkyls, such as cyclobutyl, cyclopentyl, cyclohexyl, and bicyclo[2.2.1]heptyl;

heteroaryls, such as imidazolyl(C₁-C₆)alkyl and pyridyl(C₁-C₆)alkyl;

and

heterocycles selected from acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydroquinolyl, tetrazolyl, thiadiazolyl, thiazolidinyl, thiazolyl, thienyl, thiomorpholinyl, triazolyl, bridged bicyclic groups wherein a monocyclic heterocyclic group is bridged by an alkylene group, and compounds of the formula

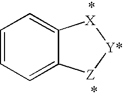

where X* and Z* are independently selected from —CH₂—, —CH₂NH—, —CH₂O—, —NH— and —O—, with the proviso that at least one of X* and Z* is not —CH₂—, and Y* is selected from —C(O)— and —(C(R″)₂)ᵥ—, where R″ is hydrogen or alkyl of one to four carbons, and v is 1-3.

In one embodiment, R₆ is selected from C₃₋₈ unsubstituted saturated cycloalkyls. In another embodiment, R₆ is selected from cyclobutyl, cyclopentyl, cyclohexyl, cycloheptyl, and bicyclo[2.2.1]heptyl, and cyclooctyl.

In one embodiment, R₆ is selected from unsubstituted heteroaryls. In another embodiment, R₆ is selected from imidazolyl(C₁-C₆)alkyl, tetrahydropyranyl(C₁-C₆)alkyl, piperidinyl(C₁-C₆)alkyl and pyridyl(C₁-C₆)alkyl.

In one embodiment, any of R₁-R₅ is selected from:

alkyl, which can be selected from alkoxyalkyl, arylalkyl, carboxyalkyl, carboxycycloalkyl, carboxycycloalkylalkyl, cycloalkylalkyl, haloalkyl, and hydroxyalkyl;

alkanoyl, which can be selected from alkanoyloxy, aminoalkanoyl, arylalkanoyl, and hydroxyalkanoyl;

alkenyl, which can be carboxyalkenyl;

alkoxy, which can be selected from alkoxyalkoxy, amidoalkoxy, aminoalkoxy, carboxyalkoxy, carboxycycloalkoxy, and hydroxyalkoxy;

aldehyde, which can be aldehyde hydrazone;

amido, which can be selected from alkylamido, alkylsulfonylamido, alkoxycarbonylamido, aminocarbonyl, arylcarboxyamido, arylsulfonylamido, carboxyamido, carboxyaminocarbonyl, and heterocyclylamido, heterocyclylsulfonylamido, hydroxyamido, sulfonylalkylamido;

amino, which can be selected from carboxyamino, heterocyclylamino, hydroxyamino;

carbonyl-containing group, which can be selected from arylalkoxycarbonyl, aryloxycarbonyl, alkenoxycarbonyl, alkoxycarbonyl, carboxycarbonyl, carboxyalkylcarbonyl, heterocyclylcarbonyl;

ester, which can be selected from alkanoyloxyalkyl;

perfluoroalkyl, which can be selected from trifluoromethyl;

sulfonyl, which can be selected from alkylsulfonyl, aminosulfonyl, arylsulfonyl, arylalkylsulfonyl, heterocyclylsulfonyl, heterocyclylalkylsulfonyl, and sulfonylalkylsulfonyl; and

thio, which can be selected from alkylthio, thioamido, and carboxythioalkoxy.

In one embodiment, R₁ and R₂ are selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, perfluoroalkyl, sulfonyl, sulfonate, thio, and other carbonyl-containing groups.

In another embodiment, R₁ and R₂ are selected from hydrogen, alkyl, halogen, haloalkyl, and nitro.

Another embodiment of the present invention provides a compound of formula I:

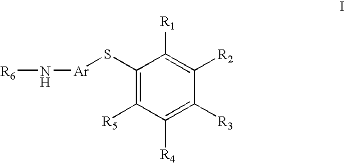

and pharmaceutically-acceptable salts and prodrugs thereof,

wherein R₁, R₂, R₃, R₄, and R₅, are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

R₆ is selected from unsubstituted alkyls, unsubstituted saturated cycloalkyls, unsubstituted saturated carboxycycloalkyls, unsubstituted carboxyalkyls, and unsubstituted heterocyclylalkyls,

wherein the unsubstituted saturated cycloalkyls, unsubstituted saturated carboxycycloalkyls, unsubstituted carboxyalkyls, and unsubstituted heterocyclylalkyls are bonded to the NH of formula I through the alkyl group,

with the proviso that the heterocyclylalkyl is not

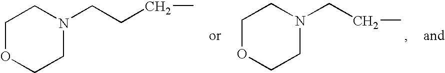

with the proviso that at least one of R₁ or R₃ is selected from:

(A) substituents of formula IV:

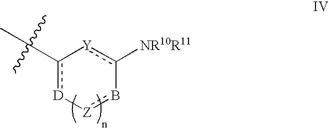

wherein D, B, Y and Z are each independently selected from the group consisting of —CR³¹═, —CR³²R³³—, —C(O)—, —O—, —SO₂—, —S—, —N═, and —NR³⁴—;

n is an integer of zero to three; and

R³¹, R³², R³³ and R³⁴ are each independently selected from the group consisting of hydrogen, alkyl, carboxy, hydroxyalkyl, alkylaminocarbonyl alkyl, dialkylaminocarbonylalkyl and carboxyalkyl; and

(B) cyclopropyl derivatives selected from cis-cyclopropanoic acid, trans-cyclopropanoic acid, cis-cyclopropanamide and trans-cyclopropanamide defined as

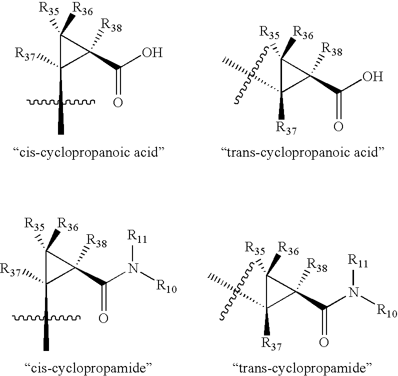

wherein R₃₅ and R₃₆ are each independently selected from the group consisting of hydrogen, alkyl, carboxy, hydroxyalkyl and carboxyalkyl, and

wherein R₃₇ and R₃₈ are each independently selected from the group consisting of hydrogen, alkyl, carboxyalkyl, alkylaminocarbonylalkyl and dialkylaminocarbonylalkyl, and

wherein R₁₀ and R₁₁ are each independently selected from hydrogen, alkanoyl, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups, or
where R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and

wherein R₁ and/or R₂, and R₄ and R₅ can be joined to form a 5- to 7-membered cycloalkyl or heterocyclyl ring when R₃ is the cinnamide, and R₂ and R₃, R₃ and R₄, and/or R₄ and R₅ can be joined to form a 5- to 7-membered ring when R₁ is the cinnamide,

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups.

Another embodiment of the present invention provides a compound of formula I:

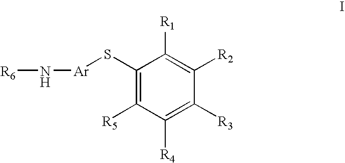

and pharmaceutically-acceptable salts and prodrugs thereof,

wherein R₁, R₂, R₃, R₄, R₅, and R₆ are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

with the proviso that at least one of R₁ or R₃ is selected from:

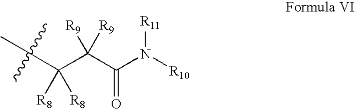

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, sulfonate, sulfonyl, thio, and other carbonyl-containing groups,

wherein R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups,

R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein R₁ and R₂, and R₄ and R₅ can be joined to form a 5- to 7-membered cycloalkyl or heterocyclyl ring when R₃ is a substituent of formula VI, and R₂ and R₃, R₃ and R₄, and R₄ and R₅ can be joined to form a 5- to 7-membered ring when R₁ is a substituent of formula VI.

Another embodiment of the present invention provides a compound of formula I:

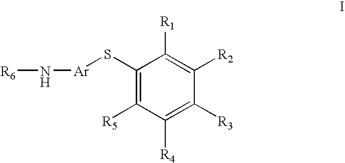

and pharmaceutically-acceptable salts and prodrugs thereof,

wherein R₁, R₂, R₃, R₄, R₅, and R₆ are independently selected from hydrogen, alkyl, alkenyl, alkenoxy, alkynyl, aldehyde, alkanoyl, alkoxy, amido, amino, aminothiocarbonyl, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

with the proviso that at least one of R₁ or R₃ is selected from cinnamic acids of formula VII:

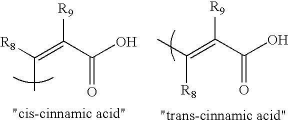

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, sulfonate, sulfonyl, thio, and other carbonyl-containing groups,

wherein R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, sulfonyl, thio, and other carbonyl-containing groups,

R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone,,nitro, oxo, perfluoroalkyl, sulfonyl, sulfonate, thio, and other carbonyl-containing groups,

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,

wherein R₁ and R₂, and R₄ and R₅ can be joined to form a 5- to 7-membered cycloalkyl or heterocyclyl ring when R₃ is a substituent of formula VII, and R₂ and R₃, R₃ and R₄, and R₄ and R₅ can be joined to form a 5- to 7-membered ring when R₁ is a substituent of formula VII.

Another embodiment of the present invention provides a compound of formula III:

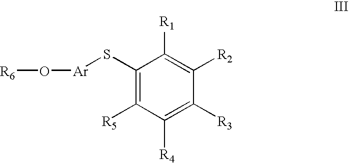

and pharmaceutically-acceptable salts and prodrugs thereof,

wherein R₁, R₂, R₃, R₄, and R₅, are independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, perfluoroalkyl, sulfonyl, sulfonate, thio, and other carbonyl-containing groups,

alternatively, R₁, R₂, R₃, R₄, R₅, and R₆ may independently be aminothiocarbonyl,

wherein R₆ is carboxycycloalkyl, with the proviso that at least one of R₁ and R₃ is cis-cinnamide or trans-cinnamide defined as

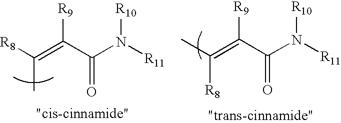

or alternatively, with the proviso that at least one of R₁ and R₃ is selected from (A) substituents of formula IV, and (B) cyclopropyl derivatives selected from cis-cyclopropanoic acid, trans-cyclopropanoic acid, cis-cyclopropanamide and trans-cyclopropanamide, as defined above,

or alternatively, with the proviso that at least one of R₁ and R₃ is selected from substituents of formula VI, as defined above,

or alternatively, with the proviso that at least one of R₁ and R₃ is selected from substituents of formula VII, as defined above,

wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, and other carbonyl-containing groups,

wherein R₁₀ and R₁₁ are each independently selected from hydrogen, alkyl, alkenyl, alkynyl, alkoxy, aryl, arylalkyl, carboxy, cyano, cycloalkyl, ester, ether, heterocyclyl, hydroxy, ketone, nitro, and other carbonyl-containing groups,

R₁₀ and R₁₁ may independently be alkanoyl, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and

wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups.

In one embodiment, the carboxycycloalkyl has a C₁₋₆ alkyl. In another embodiment, the cycloalkyl group of the carboxycycloalkyl is selected from cyclobutyl, cyclopentyl, cyclohexyl, cycloheptyl, and cyclooctyl. In yet another embodiment, R₆ is carboxycyclohexyl.

### Preparation of Compounds

Preparation of the compounds of the invention can be exemplified by the following schemes and reactions.

In one embodiment, the synthesis of the compound of formula II can be envisioned as piecing together various components A-G, as illustrated below:

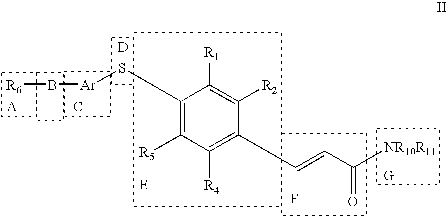

One of ordinary skill in the art will appreciate that the components A-G may be capable of assembly in any order. Component B can be, for example, NH or O. Components F and G can be prepared, for example, by activating a protected acrylic acid a with an —NR₁₀R₁₁-containing reagent to form acrylamide b, as shown in Scheme 1.

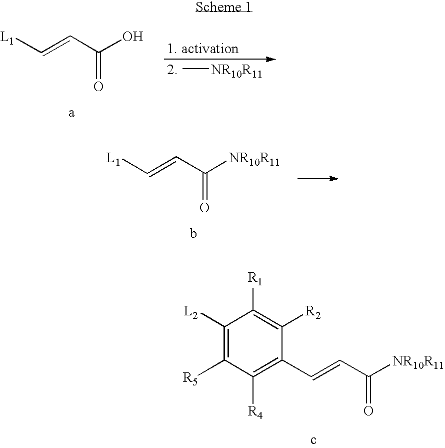

Although Scheme 1 shows the trans form of acrylamide b, one of ordinary skill in the art can appreciate that the cis or trans form can be prepared in any of the described Schemes.

Component E can be prepared by subsequent conversion of the functionalized end of b into cinnamide c. The aryl group can be substituted with any one of substituents R₁, R₂, R₄, R₅, and L₂ prior to or after reacting with b. Exemplary L₁ groups include furyl, hydrogen, triflate, and halogen (e.g., organometallic coupling reactions). Exemplary L₂ groups include hydroxy, sulfonate ester, halogen, and aryl sulfide.

Conversely, an aryl group (or aryl disulfide) can be functionalized with an acrylic acid, as in d, and subsequently reacted to form cinnamide e, as shown in Scheme 2.

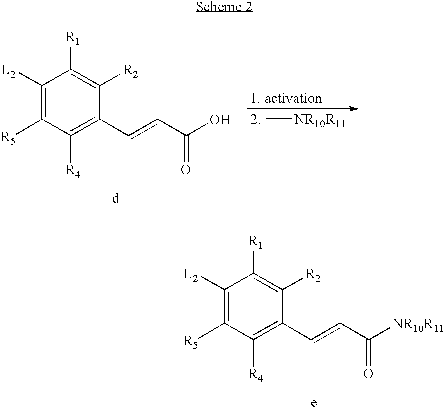

One of ordinary skill in the art will appreciate that component F may be formed simultaneously with component E, for example, by condensation of a benzaldehyde with another carbonyl containing molecule (e.g., aldol or Knoevenagel type condensations).

Components C and D, the aryl or heteroaryl sulfide, can be attached to an aryl group by reacting the aryl group with a thiol or a thiolate. Exemplary aryl sulfide-forming reactions are described in WO 00/59880, pp. 71-90, the disclosure of which is incorporated by reference herein in its entirety. Alternatively, an aryl group, such as a phenol, can be reacted with a sulfonic acid or sulfonate-containing species, to produce a corresponding aryl sulfonic acid ester, as shown in Scheme 3 below.

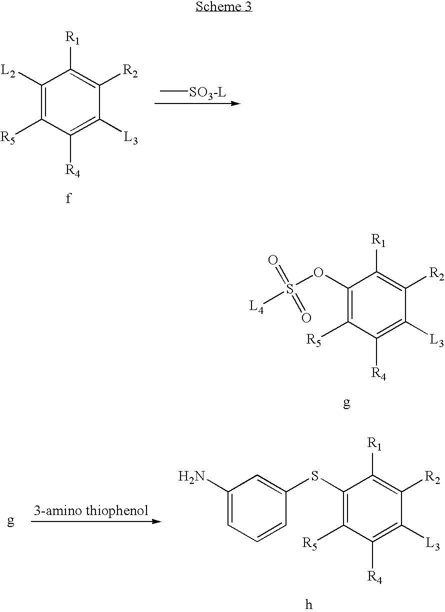

L₂ can be a hydroxy group, or any group capable of reacting with reagents containing the —SO₃-L₄ unit. Exemplary reagents containing the —SO₃-L₄ unit include trifluoromethanesulfonic acid. L₃ can be a cinnamic acid or cis or trans cinnamide or any precursor to a cinnamic acid or cinnamide.

The sulfonic acid ester g in Scheme 3 can be attached to an aryl group by reaction with, for example, a substituted or unsubstituted arylthiol, or any other reagent capable of reacting with g. Scheme 3 illustrates the reaction of sulfonic acid ester g with 3-amino thiophenol to produce the 3-aminophenylsulfanyl unit, h.

In one embodiment, R₆ can be attached by reacting the NH₂-derivative, h (prepared by, for example, Scheme 3) with an R₆-containing reagent, or an R₆ precursor. For example, R₆ can be attached by reacting h with an R₆-containing halide, carbonyl halide, oxo or ketone, ajdehyde, sulfonyl halide (such as an R₆-containing sulfonyl chloride), isocyanate, isothiocyanate, haloformate (such as chloroformate), ester, hydroxy or alcohol, carboxylic acid, and anhydride.

In one embodiment, the NH₂ group on the derivative h can be protected with a protecting group P to form protected amine NHP. The NHP derivative then can be reacted with an R₆ containing reagent or precursor to form an NR₆P derivative followed by deprotection to form the NHR₆ derivative.

In one embodiment, h can be converted to another starting material capable of reacting with an R₆-containing reagent.

In one embodiment, R₆ can be attached to component B priorto formation of the diaryl sulfide. For example, reagent g (prepared by, for example, Scheme 3) can be reacted with an R₆—N(H)-thiophenol.

Synthesis of pyrimidine derivatives (Component F of formula II) is shown in Scheme 4. L₂ is as described above. Reaction of methyl ketone i with diethylcarbonate under base catalysis leads to beta-ketoester j. Condensation of j with formamidine gives 4-hydroxypyrimidine k, which can be converted into 4-chloropyrimidine I. Displacement of the chloride of I by an amine then gives pyrimidine m.

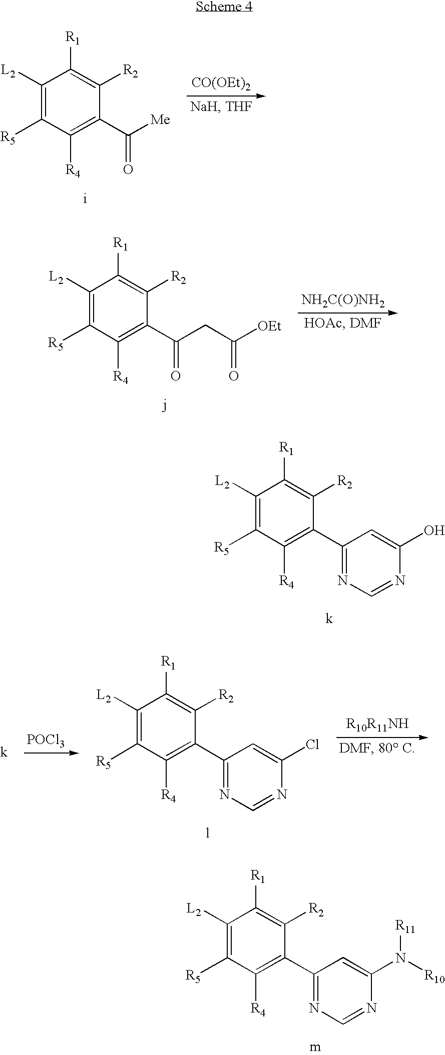

Another route to 4,6-disubstituted pyrimidines m is illustrated in Scheme 5. Transmetallation of n with n-BuLi/ZnCl₂, followed by Pd-catalyzed cross-coupling with 4,6-diiodopyrimidine leads to iodopyrimidine o. Reaction of o with selected amines gives pyrimidine m.

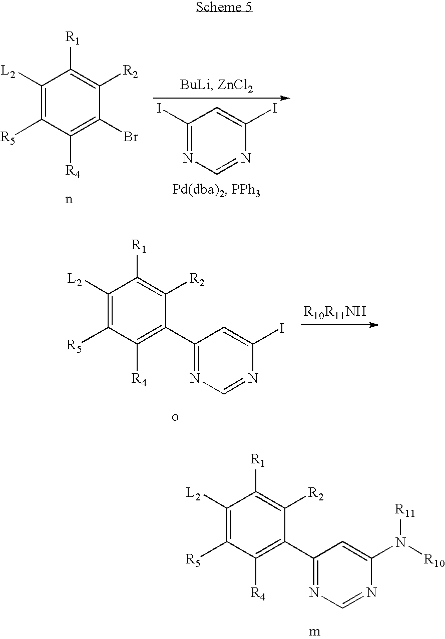

Synthesis of pyridine derivatives (Component F of formula II) can be achieved as shown in Scheme 6. Palladium-catalyzed cross-coupling of properly substituted 1-bromo-4-fluorobenzene p and 4-pyridine boronic acid gives pyridine q. Oxidation of q affords pyridinium oxide r. Fluoride displacement of r with an aryl thiol gives diarylsulfide s. Treatment of s with POCl₃ leads to 2-chloropyridine t. Finally, reaction of t with selected amines gives 2-aminopyridine u.

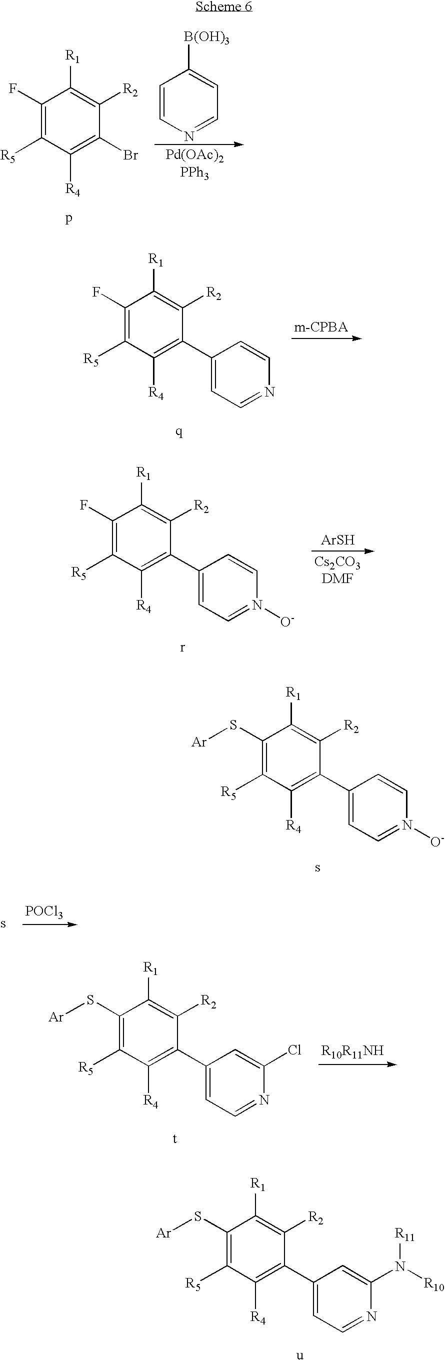

Cyclopropyl derivatives (Component F of formula II) can be accessed by the process shown in Scheme 7, wherein L₂ is as described above. Aldehyde v is treated with an acetate equivalent under basic conditions to afford ester w. Reaction of w with trimethylsulfoxonium iodide in the presence of base (e.g., NaH), followed by hydrolysis of the intermediate ester (using, e.g., NaOH in alcohol), gives cyclopropane acid x. Treatment of x with an amine yields cyclopropanamide y.

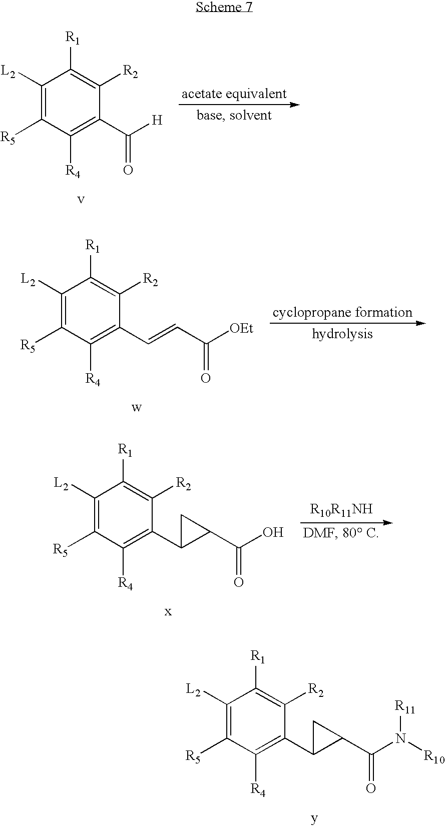

Cyclopropyl derivatives can also be prepared by palladium-mediated coupling of a halo- or trifluorosulfonyl-substituted diarylsulfide with an appropriately substituted alkene. Coupling can be achieved using, e.g., tetrakis(triphenyl phosphine)palladium (0), Pd₂(dba)₃, or the like. Cyclopropanation (using, e.g., ethyl diazoacetate and rhodium catalyst) then yields the diarylsulfide cyclopropane derivative. Direct coupling of substituted cyclopropanes with halo- or trifluorosulfonyl-substituted diarylsulfides also affords diarylsulfide cyclopropane derivatives.

Cyclopropyl, pyridine, and pyrimidine derivatives are given below in Table 1.

TABLE 1

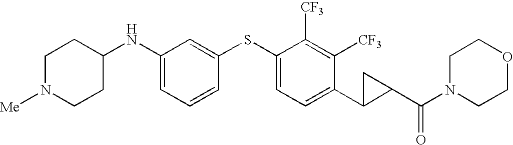

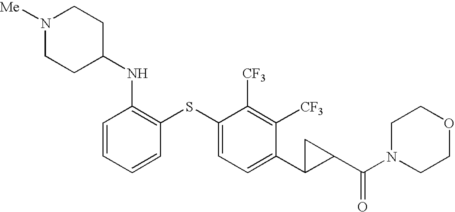

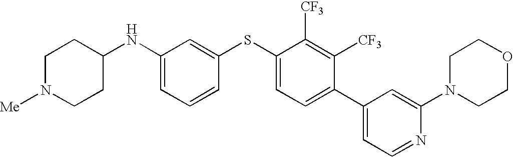

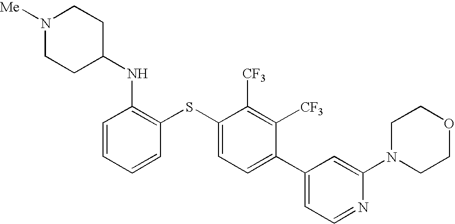

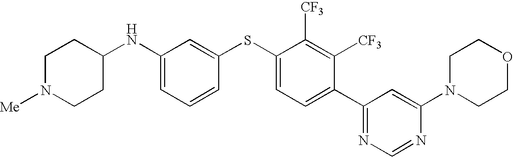

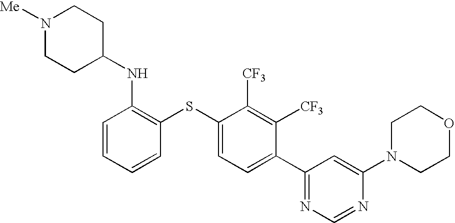

Other substitutions can be performed by the teachings of Publication Nos. WO 00/39081, WO 00/59880, WO 02/02522, and WO 02/02539, the disclosures of which are incorporated by reference herein.

Non-limiting examples of groups of Formula IV include

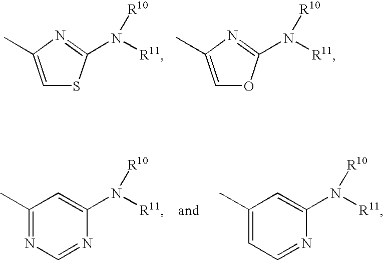

wherein R₁₀ and R₁₁ are as defined above.

### Pharmaceutical Compositions

The present invention also provides pharmaceutical compositions comprising compounds of the present invention formulated together with one or more pharmaceutically-acceptable carriers. The pharmaceutical compositions may be specially formulated for topical administration. Alternatively, the pharmaceutical compositions may be specially formulated for oral administration in solid or liquid form, for parenteral injection, for rectal administration, or for vaginal administration. The pharmaceutical compositions may encompass crystalline and amorphous forms of the active ingredient(s).

As used herein, the phrase “pharmaceutically-acceptable carrier” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

The pharmaceutical compositions of this invention can be administered to humans and other animals orally, rectally, parenterally, intracisternally, intravaginally, intraperitoneally, topically (as by powders, ointments, or drops), buccally, or as an oral or nasal spray. The compositions may also be administered through the lungs by inhalation. The term “parenteral administration” as used herein refers to modes of administration, which include intravenous, intramuscular, intraperitoneal, intracisternal, subcutaneous and intraarticular injection and infusion.

Pharmaceutical compositions of this invention for parenteral injection comprise pharmaceutically-acceptable aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, and polyethylene glycol), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservatives, wetting agents, emulsifying agents, and dispersing agents. They may also contain taggants or other anti-counterfeiting agents, which are well known in the art. Prevention of the action of microorganisms may be ensured by the inclusion of various antibacterial and antifungal agents, for example, paraben, chlorobutanol, and phenol sorbic acid. It may also be desirable to include isotonic agents such as sugars, and sodium chloride. Prolonged absorption of the injectable pharmaceutical form may be brought about by the inclusion of agents, which delay absorption such as aluminum monostearate and gelatin.

In some cases, in order to prolong the effect of the drug, it may be desirable to slow the absorption of the drug following subcutaneous or intramuscular injection. This may be accomplished by the use of a liquid suspension of crystalline or amorphous material with poor water solubility. Amorphous material may be used alone or together with stabilizers as necessary. The rate of absorption of the drug then depends upon its rate of dissolution, which in turn, may depend upon crystal size and crystalline form.

Alternatively, delayed absorption of a parenterally administered drug form can be accomplished by dissolving or suspending the drug in an oil vehicle.

Injectable depot forms can be made by forming microencapsulating matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions, which are compatible with body tissues.

The injectable formulations can be sterilized, for example, by filtration through a bacterial-retaining filter, or by incorporating sterilizing agents in the form of sterile solid compositions, which can be dissolved or dispersed in sterile water or other sterile injectable medium just prior to use.

Solid dosage forms for oral administration include capsules, tablets, pills, powders, and granules. Such forms may include forms that dissolve or disintegrate quickly in the oral environment. In such solid dosage forms, the active compound can be mixed with at least one inert, pharmaceutically-acceptable excipient or carrier. Suitable excipients include, for example, (a) fillers or extenders such as starches, lactose, sucrose, glucose, mannitol, and silicic acid; (b) binders such as cellulose and cellulose derivatives (such as hydroxypropylmethylcellulose, hydroxypropylcellulose, and carboxymethylcellulose), alginates, gelatin, polyvinylpyrrolidone, sucrose, and acacia; (c) humectants such as glycerol; (d) disintegrating agents such as sodium starch glycolate, croscarmellose, agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents, such as cetyl alcohol and glycerol monostearate, fatty acid esters of sorbitan, poloxamers, and polyethyleneglycols; (h) absorbents such as kaolin and bentonite clay; (i) lubricants such as talc, calcium stearate, magnesium stearate, solid polyethylene glycols, sodium lauryl sulfate, and mixtures thereof; anda) glidants such as talc, and silicone dioxide. Other suitable excipients include, for example, sodium citrate or dicalcium phosphate. The dosage forms may also comprise buffering agents.

Solid or semi-solid compositions of a similar type may also be employed as fillers in soft and hard-filled gelatin capsules using such excipients as lactose or milk sugar as well as high molecular weight polyethylene glycols.

Solid dosage forms, including those of tablets, dragees, capsules, pills, and granules, can be prepared with coatings and shells such as functional and aesthetic enteric coatings and other coatings well known in the pharmaceutical formulating art. They may optionally contain opacifying agents and colorants. They may also be in a form capable of controlled or sustained release. Examples of embedding compositions that can be used for such purposes include polymeric substances and waxes.

The active compounds can also be in micro-encapsulated form, if appropriate, with one or more of the above-mentioned excipients.

Liquid dosage forms include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers such as cyclodextrins, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, and perfuming agents. Other ingredients include flavorants for dissolving or disintegrating oral or buccal forms.

Suspensions, in addition to the active compounds, may contain suspending agents such as, for example, ethoxylated isostearyl alcohols, polyoxyethylene sorbitol and sorbitan esters, cellulose or cellulose derivatives (for example microcrystalline cellulose), aluminum metahydroxide, bentonite, agar agar, and tragacanth, and mixtures thereof.

Compositions for rectal or vaginal administration may be suppositories that can be prepared by mixing the compounds of this invention with suitable nonirritating excipients or carriers such as cocoa butter, polyethylene glycol or a suppository wax, that are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes can be formed by lipid monolayer, bilayer, or other lamellar or multilamellar systems that are dispersed in an aqueous medium. Any nontoxic, physiologically-acceptable and metabolizable lipid capable of forming liposomes can be used. The present compositions in liposome form can contain, in addition to a compound of the present invention, stabilizers, preservatives, and excipients. Exemplary lipids include the phospholipids and the phosphatidyl cholines (lecithins), both natural and synthetic.

Methods to form liposomes are known in the art. See, for example, Prescott, Ed., Methods in Cell Biology, Volume XIV, Academic Press, New York (1976), p. 33 et seq.

The compounds of the present invention may be used in the form of pharmaceutically-acceptable salts derived from inorganic or organic acids. By “pharmaceutically-acceptable salt” is meant those salts that are, within the scope of sound medical judgment, suitable for use in contact with the tissues of humans and lower animals without undue toxicity, irritation, and allergic response, and are commensurate with a reasonable benefit/risk ratio. Pharmaceutically-acceptable salts are well known in the art. For example, S. M. Berge, et al. describe pharmaceutically-acceptable salts in J Pharm Sci, 1977, 66:1-19. The salts may be prepared in situ during the final isolation and purification of the compounds of the invention or separately by reacting a free base function with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesdlfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates, such as dimethyl, diethyl, dibutyl and diamyl sulfates; long-chain halides such as decyl, lauryl, myristyl and stearyl chlorides, bromides and iodides; or arylalkyl halides, such as benzyl and phenethyl bromides and others. Water- or oil-soluble or -dispersible products are thereby obtained.

Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically-acceptable metal cation or with ammonia or an organic primary, secondary, or tertiary amine. Pharmaceutically-acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The pharmaceutical composition may also be administered intranasally, topically, or via inhalation. Dosage forms for topical, pulmonary, and nasal administration of a compound of this invention include powders, sprays, ointments, gels, creams, and inhalants. The active compound is mixed under sterile or non-sterile conditions with a pharmaceutically-acceptable carrier and any preservatives, buffers, or propellants that may be required. Ophthalmic formulations, eye ointments, powders and solutions are also contemplated as being within the scope of this invention.

### Methods of Treatment

One embodiment of the invention provides a method of treating a subject suffering from diseases chosen from inflammatory diseases, such as acute and chronic inflammatory diseases, and autoimmune diseases.

In one embodiment, the method comprises administering to a subject in need thereof a pharmaceutical composition comprising at least one of the compounds described herein. In one embodiment, the pharmaceutical composition can comprise any one of the compounds described herein as the sole active compound or in combination with another compound, composition, or biological material.

In one embodiment, the invention provides a method of treatment or prophylaxis in which the inhibition of inflammation or suppression of immune response is desired. In another embodiment, the method comprises suppressing an immune response comprising administering to a subject the pharmaceutical composition.

Another embodiment of the invention provides a method of treating a disease mediated at least in part by LFA-1, comprising administering a pharmaceutical composition comprising any compound described herein. In one embodiment, a “disease mediated at least in part by LFA-1” as used herein refers to a disease resulting partially or fully from LFA-1 binding.

Another embodiment of the invention provides a method of treating a disease responsive to an inhibitor of LFA-1, comprising administering a pharmaceutical composition comprising any compound described herein.

In one embodiment, a “subject” as used herein is a mammal, such as a human. In one embodiment, the subject is suspected of having an inflammatory or autoimmune disease, e.g., shows at least one symptom associated with an inflammatory or autoimmune disease. In another embodiment, the subject is one susceptible to having an inflammatory or autoimmune disease, for example, a subject genetically disposed to having the disease.

The terms “treatment,” “therapeutic method,” and their cognates refer to both therapeutic treatment and prophylactic/preventative measures. Those in need of treatment may include individuals already having a particular medical disease as well as those at risk for the disease (ire., those who are likely to ultimately acquire the disorder). A therapeutic method results in the prevention or amelioration of symptoms or an otherwise desired biological outcome and may be evaluated by improved clinical signs, delayed onset of disease, reduced/elevated levels of lymphocytes and/or antibodies, etc.

The term “immune disease” refers to disorders and conditions in which an immune response is aberrant. The aberrant response can be due to abnormal proliferation, maturation, survival, differentiation, or function of immune cells such as, for example, T or B cells.

Exemplary indications that can be treated by a method according to the invention include, but are not limited to: ischemic-reperfusion injury, such as pulmonary reperfusion injury; stroke; asthma; myocardial infarction; psoriasis, such as chronic plaque, pustular, guttate, and erythrodermic psoriasis; atherosclerosis; atopic dermatitis; hepatitis; adult respiratory distress syndrome; chronic ulceration; lung fibrosis; graft-versus-host disease; chronic obstructive pulmonary disease; Sjögren's syndrome; multiple sclerosis; autoimmune thyroiditis; glomerulonephritis; systemic lupus erythematosus; diabetes; primary biliary cirrhosis; autoimmune uveoretinitis; scleroderma; arthritis, such as psoriatic arthritis and Lyme arthritis; fulminant hepatitis; inflammatory liver injury; thyroid diseases such as Graves' disease; transplant rejection (islets, liver, kidney, heart, etc.); inflammatory lung injury; radiation pneumonitis; inflammatory bowel diseases such as Crohn's disease and ulcerative colitis; inflammatory glomerular injury; radiation-induced enteritis; peripheral artery occlusion; graft rejection; and cancer.

In one embodiment, the present invention provides a method of treatment of any of the indications listed below.

In one embodiment, the present invention provides a method of treating psoriasis. Psoriasis can manifest as one of four forms: chronic plaque, pustular, guttate, and erythrodermic. For example, the role of LFA-1 antagonism can be supported clinically with the use of the monoclonal antibody Efalizumab (Raptiva™) as a treatment for moderate to severe chronic plaque psoriasis (Lebwohl et al., N Engl J Med, 349(21): 2004-2013, 2003. Similarly, small molecule antagonists of LFA-1 may be effective treatments for psoriasis and other inflammatory and autoimmune diseases (Liu, G., Expert Opinion, 11:1383, 2001).

The role of LFA-1 antagonism in treating arthritis can be demonstrated by: a murine collagen-induced arthritis model according to the method of Kakimoto et al., Cell Immunol 142:326-337,1992; a rat collagen-induced arthritis model according to the method of Knoerzer et al., Toxicol Pathol 25:13-19,1997; a rat adjuvant arthritis model according to the method of Halloran et al., Arthritis Rheum 39:810-819,1996; a rat streptococcal cell wall-induced arthritis model according to the method of Schimmer et al., J Immunol, 160:1466-1477,1998; and a SCID-mouse human rheumatoid arthritis model according to the method of Oppenheimer-Marks et al., J Clin Invest 101:1261-1272, 1998.

The role of LFA-1 antagonism in treating fulminant hepatitis can be demonstrated by a murine model of ConA-induced acute hepatic damage (G. Matsumoto et al., J Immunol 169(12):7087-7096, 2002).

The role of LFA-1 antagonism in treating inflammatory liver injury can be demonstrated by a murine liver injury model according to the method of Tanaka et al., J Immunol 151:5088-5095, 1993.

The role of LFA-1 antagonism in treating Sjögren's syndrome can be demonstrated by the studies of Mikulowska-Mennis et al., Am J Pathol 159(2):671-681, 2001. Lymphocyte migration to inflamed lacrimal glands is mediated by vascular cell adhesion molecule-1/alpha(4)beta(1) integrin, peripheral node addressin/l-selectin, and lymphocyte function-associated antigen-1 adhesion pathways.

The role of LFA-1 antagonism in treating autoimmune thyroid diseases such as Graves' disease can be demonstrated by the studies of Arao et al., J Clin Endocrinol Metab, 85(1):382-389, 2000.

The role of LFA-1 antagonism in treating multiple sclerosis can be demonstrated by several animal models demonstrating inhibition of experimental autoimmune encephalomyelitis by antibodies to LFA-1 (E. J. Gordon et al., J Neuroimmunol 62(2):153-160, 1995). Piccio et al. also demonstrated that the firm in vivo arrest of T lymphocytes to inflamed brain venules was LFA-1 dependent (L. Piccio et al., J Immunol, 168(4):1940-1949, 2002).

The role of LFA-1 antagonism in treating autoimmune diabetes can be demonstrated by the method of Fabien et al., Diabetes 45(9):1181-1186, 1996. The role of LFA-1 antagonism in treating autoimmune diabetes can be demonstrated by an NOD mouse model according to the method of Hasagawa et al., Int Immunol 6:831-838, 1994, and by a murine streptozotocin-induced diabetes model according to the method of Herrold et al., Cell Immunol 157:489-500, 1994. Furthermore, several studies have demonstrated improvement in the rate of survival of transplanted islets upon treatment with LFA-1 antagonists (M. Nishihara et al., Transplant Proc 27(1):372, 1995; see also L. Buhler et al., Transplant Proc 26(3):1360-1361, 1994.

The role of LFA-1 antagonism in treating Lyme arthritis can be demonstrated by the method of Gross et al., Science 281:703-706,1998.

The role of LFA-1 antagonism in treating asthma can be demonstrated by a murine allergic asthma model according to the method of Wegner et al., Science 247:456-459, 1990, or in a murine non-allergic asthma model according to the method of Bloemen et al., Am J Respir Crit Care Med 153:521-529, 1996.

The role of LFA-1 antagonism in treating inflammatory lung injury can be demonstrated by: a murine oxygen-induced lung injury model according to the method of Wegner et al., Lung 170:267-279,1992; a murine immune complex-induced lung injury model according to the method of Mulligan et al., J Immunol 154:1350-1363,1995; and a murine acid-induced lung injury model according to the method of Nagase, et al., Am J Respir Crit Care Med 154:504-510,1996.

The role of LFA-1 antagonism in treating radiation pneumonitis can be demonstrated by a murine pulmonary irradiation model according to the method of Hallahan et al., Proc Natl Acad Sci USA, 94:6432-6437,1997.

The role of LFA-1 antagonism in treating inflammatory bowel disease can be demonstrated by a rabbit chemical-induced colitis model according to the method of Bennet et al., J Pharmacol Exp Ther, 280:988-1000, 1997.

The role of LFA-1 antagonism in treating inflammatory glomerular injury can be demonstrated by a rat nephrotoxic serum nephritis model according to the method of Kawasaki, et al., J Immunol, 150:1074-1083, 1993.

The role of LFA-1 antagonism in treating radiation-induced enteritis can be demonstrated by a rat abdominal irradiation model according to the method of Panes et al., Gastroenterology 108:1761-1769, 1995.

The role of LFA-1 antagonism in treating reperfusion injury can be demonstrated by the isolated rat heart according to the method of Tamiya et al., Immunopharmacology 29(1):53-63, 1995, or in the anesthetized dog according to the model of Hartman et al., Cardiovasc Res 30(1):47-54, 1995.

The role of LFA-1 antagonism in treating pulmonary reperfusion injury can be demonstrated by a rat lung allograft reperfusion injury model according to the method of DeMeester et al., Transplantation 62(10):1477-1485, 1996, and a rabbit pulmonary edema model according to the method of Horgan et al., Am J Physiol 261(5):H1578-H1584, 1991.

The role of LFA-1 antagonism in treating stroke can be demonstrated by: a rabbit cerebral embolism stroke model according the method of Bowes et al., Exp Neurol 119(2):215-219, 1993; a rat middle cerebral artery ischemia-reperfusion model according to the method of Chopp et al., Stroke 25(4):869-875, 1994; and a rabbit reversible spinal cord ischemia model according to the method of Clark et al., Neurosurg 75(4):623-627, 1991.

The role of LFA-1 antagonism in treating peripheral artery occlusion can be demonstrated by a rat skeletal muscle ischemia/reperfusion model according to the method of Gute et al., Mol Cell Biochem 179:169-187, 1998.

The role of LFA-1 antagonism in treating graft rejection can be demonstrated by: a murine cardiac allograft rejection model according to the method of Isobe et al., Science 255:1125-1127, 1992; a murine thyroid gland kidney capsule model according to the method of Talento et al., Transplantation 55:418-422, 1993; a cynomolgus monkey renal allograft model according to the method of Cosimi et al., J Immunol 144:4604-4612, 1990; a rat nerve allograft model according to the method of Nakao et al., Muscle Nerve, 18:93-102, 1995; a murine skin allograft model according to the method of Gorczynski et al., J Immunol 152:2011-2019, 1994; a murine corneal allograft model according to the method of He et al., Opthalmol. Vis Sci 35:3218-3225, 1994; and a xenogeneic pancreatic islet cell transplantation model according to the method of Zeng et al., Transplantation 58:681-689, 1994.

The role of LFA-1 antagonism in treating graft-versus-host disease (GVHD) can be demonstrated by a murine lethal GVHD model according to the method of Haming et al., Transplantation 52:842-845, 1991.

The role of LFA-1 antagonism in treating cancers can be demonstrated by a human lymphoma metastasis model (in mice) according to the method of Aoudjit et al., J Immunol 161:2333-2338, 1998.

The role of LFA-1 antagonism in treating atopic dermatitis is supported by the reports of M. Murayama et al., Arch Dermatol Res 289(2):98-103, 1997, and S. Kondo et al., Br J Dermatol 131(3):354-9, 1994.

The role of LFA-1 antagonism in treating autoimmune uveoretinitis is supported by the reports of E. Uchio et al., Invest Ophthalmol Vis Sci 35(5):2626-2631, 1994, and H. Xu et al., J Immunol 172(5):3215-3224, 2004.

The role of LFA-1 antagonism in treating transplant rejection is supported by the reports of E. K. Nakakura et al., Transplantation 62(5):547-52, 1996, and by R. L. Dedrick et al., Transpi Immunol 9(2-4):181-186, 2002.

### Dosing

Actual dosage levels of active ingredients in the pharmaceutical compositions of this invention may be varied so as to obtain an amount of the active compound(s) that is effective to achieve the desired therapeutic response for a particular patient, compositions, and mode of administration. The terms “therapeutically effective dose” and “therapeutically effective amount” refer to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment, the data obtained from the assays can be used in formulating a range of dosage for use in humans.

Generally dosage levels of about 0.1 μg/kg to about 50 mg, such as a level ranging from about 5 to about 20 mg of active compound per kilogram of body weight per day, can be administered topically, orally or intravenously to a mammalian patient. Other dosage levels range from about 1 μg/kg to about 20 mg/kg, from about 1 μg/kg to about 10 mg/kg, from about 1 μg/kg to about 1 mg/kg, from 10 μg/kg to 1 mg/kg, from 10 μg/kg to 100 μg/kg, from 100 μg to 1 mg/kg, and from about 500 μg/kg to about 5 mg/kg per day. If desired, the effective daily dose may be divided into multiple doses for purposes of administration, e.g., two to four separate doses per day. In one embodiment, the pharmaceutical composition can be administered once per day.

The following assays may be used to test compounds of this invention. Unless otherwise indicated, the reagents used in the following examples are commercially available and may be purchased from Sigma-Aldrich Company, Inc. (Milwaukee, Wis., USA) or Alfa Aesar (Ward Hill, Mass., USA).

### Assays

### ICAM-I/LFA-1 Biochemical Interaction Assay

A biochemical assay may be used to measure the ability of a compound to block the interaction between the integrin LFA-1 and its adhesion partner ICAM-1. Other functionally similar agents and ingredients from alternative sources may be substituted for those described herein.

One hundred microliters (100 μL) of a non-blocking anti-LFA-1 antibody (designated TS2/4.1.1 (ATCC)) at a concentration of 5 μg/mL in 50 mM NaHCO₃/Na₂CO₃ (pH 9.6) plate coating buffer was used to coat wells of Porvair black 96-well microtiter plates overnight at 4° C. The wells were then washed three times with wash buffer (Dulbecco's phosphate-buffered saline (D-PBS) without Ca⁺⁺ or Mg⁺⁺, 0.05% Tween™ 20) and blocked by addition of 200 μL of Superblock® (Pierce Biotechnology, Rockford, Ill.) and further incubated for 1 hour at room temperature. The wells were then washed three times in wash buffer. Recombinant LFA-1 (100 μL of 1.0 μg/mL, Lupher et al., J Immunol 167:1431-1439, 2001) in D-PBS was then added to each well. Incubation was continued for 1 hour at room temperature after which the wells are washed three times with wash buffer. Serial dilutions of compounds being assayed as ICAM-1/LFA-1 antagonists, prepared from 10 mM stock solutions in dimethyl sulfoxide (DMSO), were diluted in D-PBS, 2 mM MgCl₂, 1% Superblock®, 0.05% Tween™ 20, and 50 μL of each dilution was added to duplicate wells. Fifty microliters (50 μL) of 6.0 μg/mL biotinylated recombinant ICAM-1/lg (R&D Systems, Minneapolis, Minn.) was added to the wells and the plates were incubated at room temperature for 2 hours. The wells were then washed three times with wash buffer and 100 μL of europium-labeled Streptavidin (Wallac Oy) diluted 1:1,500 in Delfia assay buffer (Wallac Oy) are added to the wells. Incubation was allowed to proceed for 1 hour at room temperature. The wells were washed eight times with wash buffer and 100 μL of enhancement solution (Wallac Oy, cat. No. 1244-105) were added to each well. Incubation was allowed to proceed for 5 minutes with constant mixing. Time-resolved fluorimetry measurements were made by using the Victor 1420 Multilabel Counter (Wallac Oy). The percent inhibition of each candidate compound was calculated by using equation (1):

$$\% inhibition = 100 \times [ 1 - ( \frac{average OD w / compound - background}{average OD w / o compound - background )} ) ] \qquad ( 1 )$$

where “background” refers to wells that were not coated with anti-LFA-1 antibody.

Compounds of the present invention exhibited inhibitory activity in the above assay. In one embodiment, inhibitory activity was indicated by determining the compound concentration at which ICAM-1/LFA-1 interaction is inhibited by 50% (IC₅₀). In certain embodiments, the compounds of the present invention have an IC₅₀ less than or equal to about 1.0 μM, such as an IC₅₀ less than or equal to about 0.1 μM, or an IC₅₀ less than or equal to about 0.01 μM, or less than or equal to about 0.001 μM.

### Cell Adhesion Assay

Biologically relevant activity of the compounds in this invention may be confirmed by using a cell-based adhesion assay and mixed lymphocyte reaction assay.

For measurement of inhibitory activity in the cell-based adhesion assay, 96-well microtiter plates were coated with 50 μL of recombinant ICAM-1/lg (R & D Systems, Inc., Minneapolis, Minn.) at a concentration of 5.0 μg/mL in 50 mM carbonate/bicarbonate buffer, pH 9.6, overnight at 4° C. Alternately, 96-well microtiter plates can be coated with ICAM-2/lg (R & D Systems, Inc., Minneapolis, Minn.) or ICAM-3/lg (R & D Systems, Inc., Minneapolis, Minn.) to determine the potency of compounds in this invention on other known LFA-1 ligands. The wells were then washed twice with 200 μL per well of D-PBS and blocked by the addition of 100 μL of a 1% solution of bovine serum albumin in D-PBS. After a 1-hour incubation at room temperature, the wells were washed once with RPMI-1640 media containing 50% heat-inactivated fetal bovine serum (adhesion media).

To determine the compound concentration at which cell adhesion is inhibited by 50% (IC₅₀), compounds were first serially diluted in DMSO to achieve a range of compound concentrations. Each diluted DMSO stock was then added to ˜0.8 mL of Adhesion Media at a concentration 1.5-fold greater than the final desired compound concentration. The final concentration of DMSO in the ICAM-1/lg-coated plate did not exceed 0.1%. Two-hundred microliters (200 μL) of the compound diluted in Adhesion Media was added per well to replicate wells (N=3 for each compound concentration) in the microtiter plate. The wells adjacent to the outer edge of the microtiter plate were not used in the cell adhesion assay, but were instead filled with 0.3 mL of Adhesion Media. The plates were then stored at 37° C. in a humidified atmosphere containing 5% CO₂.

A suspension of JY-8 cells (an LFA-1⁺ human EBV-transformed B cell line expressing the IL-8 receptor; Sadhu et al., J Immunol 160:5622-5628, 1998) was prepared containing 0.75×10⁶ cells/mL in Adhesion Media plus 90 ng/mL of the chemokine IL-8 (Peprotech, No. 200-08M). One-hundred microliters (100 μL) of the cell suspension was then added to each well of the microtiter plate containing 200 μL of diluted compound in Adhesion Media. The microtiter plates were incubated for 30 minutes in a humidified 37° C. incubator containing 5% CO₂. The reaction was then halted by the addition of 50 μL of 14% glutaraldehyde/D-PBS, the plates covered with sealing tape (PerkinElmer, Inc., No.1450-461), and incubated for an additional 90 minutes at room temperature.

To remove non-adherent cells from the microtiter plate, the contents of the wells were gently decanted, and the wells were washed gently with dH₂O. Adherent cells were stained by the addition of 50 μL/well of a 0.5% crystal violet solution. After 5 minutes, the plates were washed by submersion in dH₂O to remove the excess crystal violet solution. Then 70 μL of dH₂O and 200 μL of 95% EtOH were added to each well to extract the crystal violet from the cells. Absorbance was measured 15-60 minutes later at 570 nm in an ELISA plate reader. The percent inhibition of a candidate compound was calculated by using equation (1) above.

All compounds of the present invention showed an IC₅₀ in this assay of no more than 10 μM.

### T Cell Proliferation Assay

A mixed lymphocyte reaction (MLR) may be used to determine the effect of small molecule antagonists of LFA-1 on T cell proliferation and activation. One-way MLRs can provide a measure of the mitogenic response of T lymphocytes from one individual to the alloantigens present on the cells of a second individual, provided they are mismatched in histocompatibility loci. This proliferative response can be initiated by the engagement of the T cell receptor and several co-stimulatory receptors present on T lymphocytes. LFA-1 is one of the co-stimulatory receptors. (See M. C. Wacholtz et al., J Exp Med 170(2):431-448, 1989; see also G. A. Van Seventer et al., J Immunol 144(12):4579-4586, 1990). The LFA-1 ligand ICAM-1 can provide a costimulatory signal for T cell receptor-mediated activation of resting T cells. (Blockade of LFA-1 by antibodies to CD11a blocks T cell activation and proliferation in a MLR. K. Inaba et al., J Exp Med 1;165(5):1403-17, 1987; G. A. Van Seventeretal., J Immunol 149(12):3872-80, 1992). Costimulation of T cell receptor/CD3-mediated activation of resting human CD4+ T cells by LFA-1 ligand ICAM-1 can involve prolonged inositol phospholipid hydrolysis and sustained increase of intracellular Ca²⁺ levels.

Experimental design of MLRs is well established. (See, e.g., Current Protocols in Immunology, Ed. John E. Colligan et al., John Wiley & Sons, 1999). Human peripheral blood mononuclear cells were isolated from ˜60 mL of blood from two different donors by using heparin as an anticoagulant (20 U/mL, final concentration). The blood was diluted three-fold with RPMI-1640 containing 25 mM Hepes (pH 7.4), 2 mM L-glutamine, 2 g/L sodium bicarbonate, 10 U/mL penicillin G, and 10 μg/mL streptomycin. In 50 mL polypropylene centrifuge tubes, aliquots of approximately 25 mL of diluted blood were layered onto 12.5 mL of Histopaque®-1077 (Sigma Corp., No. 1077) and the tubes were centrifuged at 514×g for 30 minutes at room temperature without braking. After centrifugation, the buffy coat containing the peripheral blood mononuclear cells was transferred to a new 50 mL tube and diluted approximately five-fold with RPM-1640 and mixed by gentle inversion. Tubes were then centrifuged at 910×g for 10 minutes at room temperature. The supernatant was aspirated, and the cells were re-suspended in MLIR media (RPMI-1640 containing 50% fetal bovine serum (HyClone), 25 mM Hepes (pH 7.4), 2 mM L-glutamine, 2 g/L sodium bicarbonate, 10 U/mL penicillin G, and 10 μg/mL streptomycin) and adjusted to a final concentration of 2×10⁶ cells/mL.

To allow for a one-way proliferative response, the cells from one blood donor (referred to as “the donor”) were irradiated with approximately 1500 rad emitted from a ¹³⁷Cs source (Mark I Irradiator, Shepard and Associates). Irradiated cells remained viable during the course of the MLR but did not proliferate in response to alloantigens. Non-irradiated cells from a second blood donor (referred to as “the responder”) were added 1:1 (50 μL:50 μL) with irradiated cells from the donor to a 96-well round-bottom microtiter plate. Each well also contained 100 μL of either LFA-1 inhibitor or MLR media alone in the case of the positive control. A negative control, designed to represent an autologous antigen response, of 50 μL of irradiated responder cells and 50 μL of non-irradiated responder cells was also present on each MLR plate.

LFA-1 inhibitors, e.g., anti-CD11a antibodies or small-molecule antagonists, were prepared at twice their final desired concentration in MLR media. Small molecule antagonists were typically tested at final concentrations ranging from 10 to 0.002 μM. Anti-CD11a monoclonal antibodies were typically tested at final concentrations ranging from 2,000 to 16 ng/mL. Six replicate wells were used for each concentration of LFA-1 inhibitor. The wells adjacent to the outer edges of the microtiter plate were not used for a MLR, but were instead filled with 200 μL of MLR media. The assay plates were then incubated at 37° C. in a 5% CO₂ atmosphere.

For each inhibitor that was tested, three identical MLR plates were prepared. The supernatants from two plates were harvested on days three and five following initiation of the MLR for cytokine analysis. The supernatant from each of the six replicate wells harvested on either day three or day five was pooled and stored at −70° C. in a 96-deepwell polypropylene plate covered with a silicone gasket. To assess T cell proliferation on the third MLR plate, 1 μCi of ³H-thymidine (New England Nuclear, No. NET-027) in 20 μL of MLR media was added per well of the MLR microtiter plate on day four. Twenty-four hours later, the cells from each well were harvested onto glass fiber filter plates (PerkinElmer Unifilter-96 GF/C plates, No. 6005147) using a Packard FilterMate Harvester (Packard Instrument Co.). ³H-Thymidine incorporation was measured as counts per minute (cpm) in a scintillation counter (Packard TopCount-NXT™).

The mean cpm from 6 replicate wells was determined for each inhibitor concentration, as well as positive (allogeneic MLR) and negative (autologous MLR) controls. The mean cpm obtained from the autologous MLRs was designated as background counts, and was subtracted from the mean cpm obtained from the positive control and LFA-1 inhibitor samples. The percent proliferation is normalized to the mean cpm obtained in the absence of inhibitor, i.e., the allogeneic MLR by using equation (2):

$$\% proliferation = 100 \times \frac{( mean inhibitor cpm - mean background cpm )}{( mean positive control cpm - mean background cpm )} \qquad ( 2 )$$

In one embodiment, the potency of the compound is indicated by determining the compound concentration at which cell proliferation is inhibited by 80% (EC₈₀). In one embodiment, wherein upon subjecting the compound to a T cell proliferation assay, the compound exhibits an EC₈₀ of less than or equal to about 3.0 μM, such as an EC₈₀ of less than or equal to about 0.3 μM or an EC₈₀ of less than or equal to about 0.03 μM.

Cytokine measurements, e.g., IL-2, IFN-γ, and TNF-α, were also determined on MLR supernatants harvested on day 3 (IL-2) and day 5 (IFN-α and TNF-α). Cytokine concentrations were determined by using ELISA kits (Biosource International) based on standard curves generated with purified cytokine standards diluted in MLR media. The background level of cytokine production was established as the mean cytokine concentration of the autologous MLR. The mean cytokine concentration of the allogeneic MLR in the absence of inhibitor was used as the positive control. The level of cytokine present in the inhibitor-treated MLRs relative to the positive control represented the percent maximal response and was calculated by using equation (3):

$$\% Maximal response = 100 \times \frac{( mean inhibitor cytokine conc . - \\ mean background cytokine conc . )}{( mean positive control conc . - cytokine mean background cytokine conc . )}$$

### EXAMPLE 1

### 3-Furan-2-yl-1-morpholin-4-yl-propenone

Furylacrylic acid (25 g, 181 mmol) was added to 200 mL of methylene chloride and the reaction was cooled to 0C. Thionyl chloride (19.8 mL, 272 mmol) was then added over 15 minutes. The solution was allowed to warm to room temperature overnight and the reaction went from cloudy to clear the next morning. In a separate flask 150 mL of methylene chloride and morpholine (47.5 mL, 545 mmol) were added and the flask was brought to 0° C. The solution containing the furan was then added dropwise by addition funnel to the cooled solution containing the morpholine. After addition the solution was allowed to warm to room temperature and stir for 1.5 hr. The reaction was then extracted twice with 1 N HCl, twice with brine, and dried over sodium sulfate. The organic layer was then decolorized by carbon and concentrated to dryness. This yielded a pale yellow solid (87% 32.5 g, 156 mmol). ¹H NMR (CDCl₃, 300 MHz) δ 3.60-3.78 (m, 8H), 6.48 (q, J=2 Hz, 1H), 6.58 (d, J=3 Hz, 1H), 6.78 (d, J=16 Hz, 1H), 7.45-7.53 (m, 2H); MS (ESI (+)) m/z 208.1 (M+H+).

### EXAMPLE 2

### 3-(4-Hydroxy-2,3-bis-trifluoromethyl-phenyl)-1-morpholin-4-yl-propenone

A solution of 3-Furan-2-yl-1-morpholin-4-yl-propenone (32 g, 106 mmol) in 80 mL of dichloroethane was prepared and placed in a Parr stirred reactor. The reactor was cooled to −78° C. and 1,1,1,4,4,4-hexafluoro-2-butyne (50 g, 219 mmol) gas was added. The was allowed to come to room temperature over two hours then the reaction was heated to 115° C. for 23 hr. HPLC analysis showed the disappearance of the starting material. The dichloroethane solution was then concentrated and brought up in 180 mL of dichloroethane. Boron trifluoride diethyl etherate (29.65 mL, 234 mmol) was added to the reaction and refluxed for three hours. The crude was concentrated and purified by column chromatography using 2:3 ethyl acetate/hexanes (47%, 27 g, 73 mmol). ¹H NMR (CDCl₃, 300 MHz) δ 3.60-3.78 (m, 8H), 6.47 (d, J=15 Hz, 1H), 7.08 (d, J=8 Hz, 1H), 7.44 (d, J=8 Hz, 1H), 7.73-7.84 (m, 1H).

### EXAMPLE 3

### Trifluoro-methanesulfonic acid 4-(3-mornholin-4-yl-3-oxo-propenyl)-2,3-bis-trifluoromethyl-phenyl ester

3-(4-Hydroxy-2,3-bis-trifluoromethyl-phenyl)-1-morpholin-4-yl-propenone (8.8 g, 23.8 mmol) was dissolved in 100 mL of dichloromethane and 6 mL of pyridine was added. The reaction was cooled to 0° C. and triflic anhydride was added slowly. After warming to room temperature the reaction was washed twice with cold 1 N HCl, twice with a cold saturated bicarbonate solution, and then dried with sodium sulfate, filtered and concentrated. (80%, 9.2 g). ¹H NMR (CDCl₃, 300 MHz) δ 3.57-3.78 (m, 8H), 6.66 (d, J=15 Hz, 1H), 7.65 (d, J=8 Hz, 1H), 7.78 (d, J=8 Hz, 1H), 7.85-7.93 (m, 1H).

### EXAMPLE 4

### 3-[4-(3-Amino-phenylsulfanyl)-2,3-bis-trifluoromethyl-phenyl]-1-morpholin-4-yl-propenone

3-Amino thiophenol (2.75 mL, 25.7 mmol) was dissolved in 86 mL of tetrahydrofuran (THF) and placed at −17° C. Lithium t-butoxide (2.0 g, 25.7 mmol) was added and the reaction was allowed to warm to room temperature before being placed back at 0° C. In a separate round bottom flask, trifluoro-methanesulfonic acid 4-(3-morpholin-4-yl-3-oxo-propenyl)-2,3-bis-trifluoromethyl-phenyl ester was dissolved in 53 mL of THF and placed at −78° C. The deprotonated 3-amino-thiophenol was then cannulated into the round bottom flask containing trifluoro-methanesulfonic acid 4-(3-morpholin-4-yl-3-oxo-propenyl)-2,3-bis-trifluoromethyl-phenyl ester at −78° C. After one hour at −78° C. the starting material was consumed. The reaction was concentrated and purified by column chromatography using 2% MeOH/98% dichloromethane (DCM) (61%, 5.21 g). ¹H NMR (DMSO-d₆, 300 MHz) δ 3.57-3.75 (m, 8H), 5.45 (s, 2H), 6.70-6.74 (m, 3H), 7.18 (t, J=8 Hz, 1H), 7.23 (d, J=15 Hz, 1H), 7.36 (d, J=9 Hz, 1H), 7.65-7.75 (m, 1H), 8.05 (d, J=9 Hz, 1H); MS (ESI (+)) m/z 477.3 (M+H+).

### EXAMPLE 5

### 3-[4-(3-Methylamino-Phenylsulfanyl)-2,3-bis-trifluoromethyl-phenyl]-1-morpholin-4-yl-propenone

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 240 μL of dimethylformamide (DMF) then methyl iodide (10.61 μL, 0.26 mmol) and potassium carbonate (14 mg, 0.10 mmol) were added. The reaction proceeded very slowly at room temperature to about 50% conversion over three days. 40% was monomethylated and 10% was dimethylated. The crude reaction was diluted with DMF and purified by preparative HPLC to give the pure mono-methylated product. MS (ESI (+)) m/z 491.1 (M+H+).

### EXAMPLE 6

### Cis 4-{3-[4-(3-morpholin-4-yl-3-oxo-propenyl)-2,3-bis-trifluoromethyl-phenylsulfanyl]-phenylamino}-cyclohexanecarboxylic acid

The product of Example 4, 3-[4-(3-amino-phenylsulfanyl)-2,3-bis-trifluoromethyl-phenyl]-1-morpholin-4-yl-propenone (1.5 g, 3.15 mmol), was dissolved in 27 mL of dichloroethane and 1,1 mL of acetic acid was added. Ethyl 4-oxocyclohexanecarboxylate (1.6 mL, 9.45 mmol) then sodium triacetoxyborohydride (2.67 g, 12.6 mmol) were added and the reaction was allowed to stir overnight. HPLC analysis showed the appearance of the two product peaks in a 3:7 ratio. The reaction product was extracted twice with sodium bicarbonate and twice with brine before drying with magnesium sulfate and concentration to give a yellow oil. The oil was dissolved in DMSO and Preparative HPLC was utilized to separate the two isomers. Each isomer was then hydrolyzed in 2:1 THF/H₂O by adding 2N LiOH until basic. The individual solutions were then concentrated and brought up in water. 1 N HCL was then added until the pH reached approximately 4 and this resulted in the precipitation of the product. The product was then filtered and washed several times with water. The isomeric products were identified as cis and trans about the cyclohexane ring by solving X-ray cocrystal structures with LFA-1. The cis compound elutes last on the HPLC and is the major product. Cis: ¹H NMR (CDCl₃, 300 MHz) δ 1.56-2.07 (m, 8H), 2.59 (m, 1H), 3.45 (m, 1H), 3.52-3.78 (m, 8H), 6.57 (d, J=16 Hz, 1H), 6.63-6.86 (m, 2H), 7.17-7.27 (m, 2H), 7.41 (d, J=9 Hz, 1H), 7.80-7.89 (m, 1H); MS (ESI (+)) m/z 603.5 (M+H⁺).

### EXAMPLE 7

### Trans 4-{3-[4-(3-morpholin-4-yl-3-oxo-propenyl)-2,3-bis-trifluoromethyl-phenylsulfanyl]-phenylamino}-cyclohexanecarboxylic acid

The procedure of Example 6 was used to prepare the Trans isomer, which eluated on the HPLC as the minor product. Trans: ¹H NMR (CDCl₃, 300 MHz) δ 1.26 (m, 2H), 1.56 (m, 2H), 2.15 (m, 4H), 2.35 (m, 1H), 3.25 (m, 1H), 3.57-3.78 (m, 8H), 6.57 (d, J=15 Hz, 1H), 6.80-6.99 (m, 2H), 7.24-7.32 (m, 2H), 7.41 (d, J=9 Hz, 1H), 7.80-7.89 (m, 1H); MS (ESI (+)) m/z 603.5 (M+H⁺).

### EXAMPLE 8

### 3-[4-(3-Cyclobutylamino-phenylsulfanyl)-2,3-bis-trifluoromethyl-phenyl]-1-morpholin-4-yl-propenone

The product of Example 4, 3-[4-(3-amino-phenylsulfanyl)-2,3-bis-trifluoromethyl-phenyl]-1-morpholin-4-yl-propenone (25 mg, 0.052 mmol), was dissolved in 450 μL of dichloroethane and 19 μL of acetic acid was added. Cyclobutanone (11.6 μL, 0.16 mmol) then sodium triacetoxyborohydride (44 mg, 0.208 mmol) were added and the reaction was allowed to stir overnight. The crude reaction mixture was diluted with DMSO and purified by preparative HPLC as the trifluoroacetamide (TFA) salt. ¹H NMR (DMSO-d₆, 300 MHz) δ 1.65-1.85 (m, 4H), 2.26-2.35 (m, 2H), 3.53-3.71 (m, 8H), 3.82 (m, 1H), 6.59-6.65 (m, 2H), 6.68 (d, J=8 Hz, 1H), 7.17-7.23 (m, 2H), 7.68 (m, 1H), 8.03 (d, J=8 Hz, 1H); MS (ESI (+)) m/z 531.3 (M+H⁺).

### EXAMPLE 9

### 4-{3-[4-(3-Morpholin-4-yl-3-oxo-prolenyl)-2,3-bis-trifluoromethyl-phenylsulfanyl]-phenylamino}-pentanoic acid

The procedure from Example 6 was followed utilizing 4-oxo-pentanoic acid ethyl ester as the starting ketone. MS (ESI (+)) m/z 591.6 (M+H⁺).

### EXAMPLE 10

### 3-(4-{3-[(1H-Imidazol-2-ylmethyl)-amino]-phenylsulfanyl}-2,3-bis-trifluoromethyl-phenyl)-1-morpholin-4-yl-propenone

The procedure from Example 8 was followed utilizing (1H-imidazol-2-yl)-acetaldehyde as the starting aldehyde. MS (ESI (+)) m/z 557.1 (M+H⁺).

### EXAMPLE 11

### 1-Morpholin-4-yl-3-(4-{3-[(pyridin-4-ylmethyl)-amino]-phenyisulfanyl}-2,3-bis-trifluoromethyl-phenyl)-propenone

The procedure from Example 8 was followed utilizing pyridin-4-yl-acetaldehyde as the starting aldehyde. MS (ESI (+)) m/z 568.5 (M+H⁺).

### EXAMPLE 12

### 1-Morpholin-4-yl-3-(4-{3-[(pyridin-3-ylmethyl)-amino]-phenylsulfanyl}-2,3-bis-trifluoromethyl-phenyl)-propenone

The procedure from Example 8 was followed utilizing pyridin-3-yl-acetaldehyde as the starting aldehyde. MS (ESI (+)) m/z 568.4 (M+H⁺).

### EXAMPLE 13

### 1-Morpholin-4-yl-3-(4-{3-[(pyridin-2-ylmethyl)-amino]-phenylsulfanyl}-2,3-bis-trifluoromethyl-phenyl)-propenone

The procedure from Example 8 was followed utilizing pyridin-2-yl-acetaldehyde as the starting aldehyde. MS (ESI (+)) m/z 568.4 (M+H⁺).

### EXAMPLE 14

### 3-[4-(3-Cyclopentylamino-phenylsulfanyl)-2,3-bis-trifluoromethyl-phenyl]-1-morpholin-4-yl-propenone

The procedure from Example 8 was followed utilizing cyclopentanone as the starting ketone. MS (ESI (+)) m/z 545.3 (M+H⁺).

### EXAMPLE 15

### 3-{4-[3-(Bicyclo[2.2.1]hept-2-ylamino)-phenylsulfanyl]-2,3-bis-trifluoromethyl-phenyl}-1-morpholin-4-yl-propenone

The procedure from Example 8 was followed utilizing bicyclo[2.2.1]heptan-2-one as the starting ketone. MS (ESI (+)) m/z 571.4 (M+H⁺).

### EXAMPLE 16

### 3-[4-(3-Cyclohexylamino-phenylsulfanyl)-2,3-bis-trifluoromethyl-phenyl]-1-morpholin-4-yl-propenone

The procedure from Example 8 was followed utilizing cyclohexanone as the starting ketone. MS (ESI (+)) m/z 545.3 (M+H⁺).

### EXAMPLE 17

### 3-[4-(2-Hydroxy-phenylsulfanyl)-2,3-bis-trifluoromethyl-phenyl]-1-morpholin-4-yl-propenone

Trifluoro-methanesulfonic acid 4-(3-morpholin-4-yl-3-oxo-propenyl)-2,3-bis-trifluoromethyl-phenyl ester (0.96 g, 1.9 mmol, Example 3) was azeotroped twice with toluene, and then dissolved in 5 mL of acetone. Potassium carbonate (0.37 g, 2.7 mmol) was dried by heating under vacuum, and then added to an acetone solution of 2-hydroxythiophenol (0.35 g, 2.8 mmol in 5 mL of acetone). To this mixture was added the triflate solution, followed by heating at reflux overnight. The reaction was concentrated, then partitioned between ethyl acetate and 1 N aqueous hydrochloric acid. The organic layer was washed with saturated aqueous sodium chloride, dried with sodium sulfate, filtered and concentrated. The residue was purified by column chromatography using 1:3-3:1 ethyl acetatelhexanes (18%, 161 mg). ¹H NMR (CDCl₃, 300 MHz) δ 3.55-3.71 (m, 8H), 6.53 (d, J=15.4 Hz, 1H), 6.99 (d, J=8.5 Hz, 1H), 7.02 (td, J=7.8, 1.2 Hz), 7.11 (dd, J=1.3,8.4 Hz, 1H), 7.40 (d, J=8.5 Hz, 1H), 7.47 (ddd, J=1.8,7.5,8.4 Hz, 1H), 7.52 (dd, J=1.8,7.5 Hz, 1H), 7.83 (dq, J=14.3,4.2 Hz, 1H); MS (ESI (+)) m/z 478.0 (M+H⁺)

### EXAMPLE 18

### 3-[4-(3-Hydroxy-phenylsulfanyl)-2,3-bis-trifluoromethyl-phenyl]-1-morpholin-4-yl-propenone

The procedure of Example 17 was followed utilizing 3-hydroxythiophenol as the starting thiophenol. MS (ESI (+)) m/z 478.0 (M+H⁺)

### EXAMPLE 19

### trans-4-{2-[4-(3-Morpholin-4-yl-3-oxo-propenyl)-2,3-bis-trifluoromethyl-phenylsulfanyl]-phenoxy}-cyclohexanecarboxylic acid

3-[4-(2-hydroxy-phenylsulfanyl)-2,3-bis-trifluoromethyl-phenyl]-1-morpholin-4-yl-propenone (51 mg, 0.11 mmol, Example 17), cis-4-hydroxy-cyclohexanecarboxylic acid methyl ester (68 mg, 0.43 mmol), and triphenylphosphine (117 mg, 0.45 mmol) were dissolved in THF (1.25 mL). Diisopropylazodicarboxylate (0.084 mL, 0.43 mmol) was added, and the solution stirred overnight at 80° C. in a sealed tube. The reaction was evaporated to dryness, and purified by preparative HPLC to give the ether. This material (48 mg, 0.078 mmol) was dissolved in THF (1.5 mL) and MeOH (1.5 mL). LiOH (1.5 mL, 2N) was added and the reaction stirred for three hours. The reaction was evaporated to dryness, then partitioned between ethyl acetate and 1 N hydrochloric acid. The organic layer was washed with saturated sodium chloride, dried with sodium sulfate, filtered and evaporated. The residue was purified by preparative HPLC to give the product (36%, 24 mg). ¹H NMR (DMSO-d₆, 300 MHz) δ 1.00 (m, 2H), 1.41 (m, 2H), 1.72 (m, 4H), 2.03 (m,1H), 3.50-3.70 (m, 8H), 4.30 (m, 1H), 7.02 (t, J=7.7Hz, 1H), 7.15 (d, J=15.0 Hz, 1H), 7.16 (d, J=8.3 Hz, 1H), 7.22 (d, J=8.3 Hz, 1H), 7.45 (td, J=8.0,1.8 Hz, 1H), 7.58 (dd, J=1.7,8.0 Hz, 1H), 7.66 (dq, J=15.1,4.4 Hz, 1H), 7.95 (d, J=8.4 Hz, 1H).

### EXAMPLE 20

### trans-4-{3-[4-(3-Morpholin-4-yl-3-oxo-propenyl)-2,3-bis-trifluoromethyl-phenylsulfanyl]-phenoxy}-cyclohexanecarboxylic acid

The procedure for Example 19 was followed utilizing 3-[4-(3-hydroxy-phenylsulfanyl)-2,3-bis-trifluoromethyl-phenyl]-1-morpholin-4-yl-propenone (Example 18) as the starting phenol. MS (ESI (+)) m/z 603.9 (M+H+).

### EXAMPLE 21

### 4-{3-[4-(3-Morpholin-4-yl-3-oxo-propenyl)-2,3-bis-trifluoromethyl-phenylsulfanyl-phenoxy}-cyclohexanecarboxylic acid

The procedure for Example 19 was followed utilizing 3-[4-(3-hydroxy-phenylsulfanyl)-2,3-bis-trifluoromethyl-phenyl]-1-morpholin-4-yl-propenone (Example 18) as the starting phenol and 4-hydroxy-cyclohexanecarboxylic acid methyl ester as the starting alcohol. MS (ESI (+)) m/z 604.2 (M+H⁺).

### EXAMPLE 22

### 4-{2-[4-(3-Morpholin-4-yl-3-oxo-propenyl)-2,3-bis-trifluoromethyl-phenylsulfanyl]-phenoxy}-cyclohexanecarboxylic acid

The procedure for Example 19 was followed utilizing 4-hydroxy-cyclohexanecarboxylic acid methyl ester as the starting alcohol. MS (ESI (+)) m/z 604.0 (M+H⁺).

### EXAMPLE 23

### cis-4-{2-[4-(3-Morpholin-4-yl-3-oxo-propenyl)-2,3-bis-trifluoromethyl-phenylsulfanyl]-phenoxy}-cyclohexanecarboxylic acid

Cis-4-hydroxy-cyclohexanecarboxylic acid (1.04 g, 7.2 mmol) and dimethylformamide di-tert-butyl acetal (5.0 mL, 20.9 mmol) were dissolved in benzene (6 mL) and heated overnight at 80° C. Isolation by aqueous workup gave cis-4-hydroxy-cyclohexanecarboxylic acid tert-butyl ester. The tert-butyl ester (0.51 g, 2.5 mmol), p-nitrobenzoic acid (1.94 g, 11.6 mmol) and triphenylphosphine (3.33 g, 12.7 mmol) were dissolved in benzene (30 mL). Diisopropylazodicarboxylate (2.5 mL, 12.7 mmol) was added, and the solution stirred overnight at 80° C. The reaction was evaporated to dryness, and purified by preparative HPLC to give the diester, 4-nitro-benzoic acid trans-4-tert-butoxycarbonyl-cyclohexyl ester (32%, 283 mg). The diester (142 mg, 0.41 mmol) was dissolved in THF (2 mL) and MeOH (2 mL). LiOH.(2 mL, 2N) was added and the reaction stirred for thirty minutes. The reaction was evaporated to dryness, then partitioned between ethyl acetate and 5% citric acid. The organic layer was washed twice with saturated sodium bicarbonate, then with saturated sodium chloride. The organic layer was dried with sodium sulfate, filtered and evaporated. The residue was purified by preparative HPLC to give trans-4-hydroxy-cyclohexanecarboxylic acid tert-butyl ester (105%, 85 mg). 3-[4-(2-Hydroxy-phenylsulfanyl)-2,3-bis-trifluoromethyl-phenyl]-1-morpholin-4-yl-propenone (50 mg, 0.10 mmol, Example 17), trans-4-hydroxy-cyclohexanecarboxylic acid tert-butyl ester (42 mg, 0.21 mmol), and triphenylphosphine (70 mg, 0.27 mmol) were dissolved in THF (1.5 mL). Diisopropylazodicarboxylate (0.042 mL, 0.21 mmol) was added, and the solution stirred overnight at 80° C. in a sealed tube. HPLC showed little conversion. Diisopropylazodicarboxylate (0.042 mL, 0.21 mmol) was added, and the solution stirred overnight at 80° C. The reaction was evaporated to dryness, and purified by preparative HPLC. This material (34 mg, 0.052 mmol) was dissolved in methylene chloride (1 mL). Trifluoroacetic acid (1 mL) was added and the reaction stirred for 1.5 h. The reaction was evaporated to dryness, and the residue was purified by preparative HPLC to give the product (24%, 7.4 mg). ¹H NMR (DMSO-d₆, 300 MHz) δ 1.32-1.61 (m, 8H), 2.19 (m, 1H), 3.49-3.70 (m, 8H), 4.52 (m, 1H), 7.03 (td, J=7.3,0.9 Hz, 1H), 7.10-7.18 (m, 2H), 7.12 (d, J=14.9 Hz, 1H), 7.47 (td, J=7.8,1.5 Hz, 1H), 7.56 (dd, J=1.8,7.7 Hz, 1H), 7.66 (dq, J=15.2,4.4 Hz, 1H), 7.92 (d, J=8.7 Hz, 1H); MS (ESI (+)) m/z 604.3 (M+H⁺).

### EXAMPLE 24

### cis-4-{3-[4-(3-Morpholin-4-yl-3-oxo-propenyl)-2,3-bis-trifluoromethyl-phenylsulfanyl]-phenoxy}-cyclohexanecarboxylic acid

The procedure for Example 23 was followed utilizing 3-[4-(3-hydroxy-phenylsulfanyl)-2,3-bis-trifluoromethyl-phenyl]-1-morpholin-4-yl-propenone (Example 18) as the starting phenol. MS (ESI (+)) m/z 604.4 (M+H⁺).

### EXAMPLE 25

### 1-Morpholin-4-yl-3-(4-{3-[(tetrahydro-pyran-4-ylmethyl)-amino]-phenylsulfanyl}-2,3-bis-trifluoromethyl-phenyl)-propenone

The product of Example 4 was subjected to procedure described in Example 8 utilizing 4-tetrahydro-pyran-4-carbaldehyde in place of cyclobutanone to afford the final product. MS (ESI (+)) m/z 575 (M+H⁺).

### EXAMPLE 26

### 1-Morpholin-4-yl-3-(4-{3-[(piperidin-4-ylmethyl)-amino]-phenylsulfanyl}-2,3-bis-trifluoromethyl-phenyl)-propenone

The procedure for Example 8 was followed substituting 4-formyl-piperidine-1-carboxylic acid tert-butyl ester for cyclobutanone. The product was dissolved in dichloromethane to which trifluoroacetic acid was added in molar excess. After one hour the reaction was concentrated to give the final product. MS (ESI (+)) m/z 574 (M+H⁺).

### EXAMPLE 27

### {3-[4-(3-Moripholin-4-yl-3-oxo-propenyl)-2,3-bis-trifluoromethyl-phenylsulfanyl]-phenylamino}-acetic acid

Product of Example 4 was reacted with bromo-acetic acid in dioxane-water (5:2) solvent at 80° C. for 3 h to afford the final product that was purified by HPLC. MS ESI (+) m/z 535 (M+H⁺).

The structure of the product compound obtained in each example is given below.

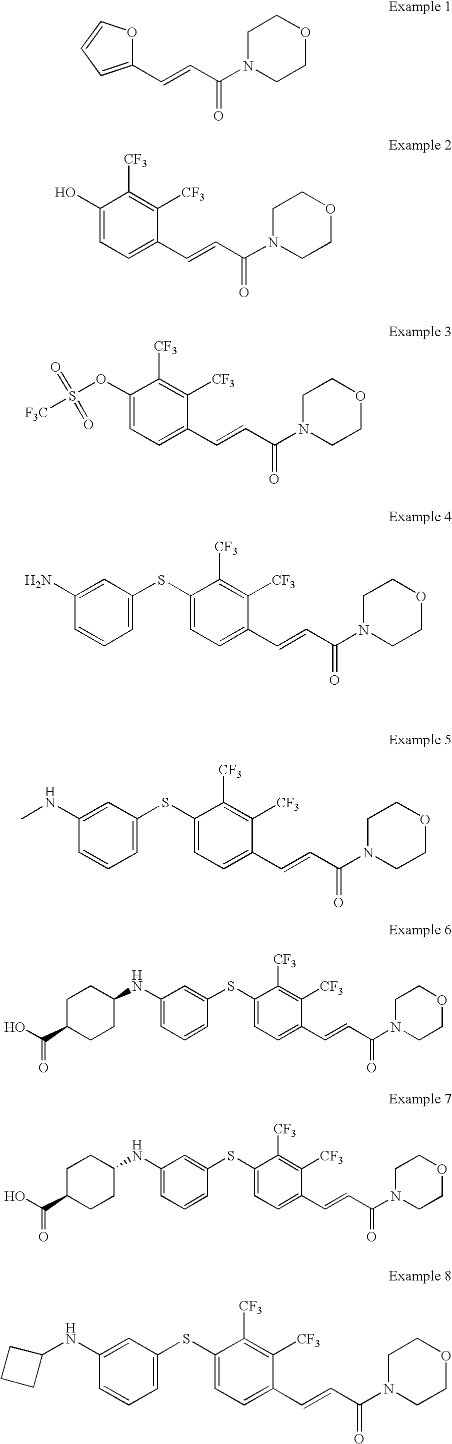

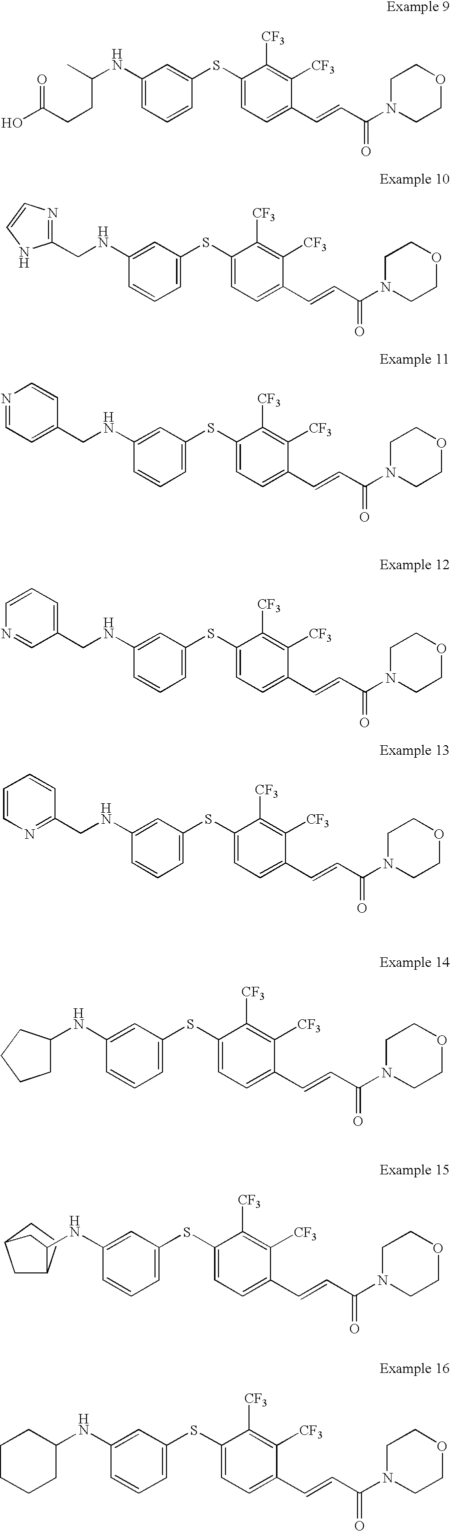

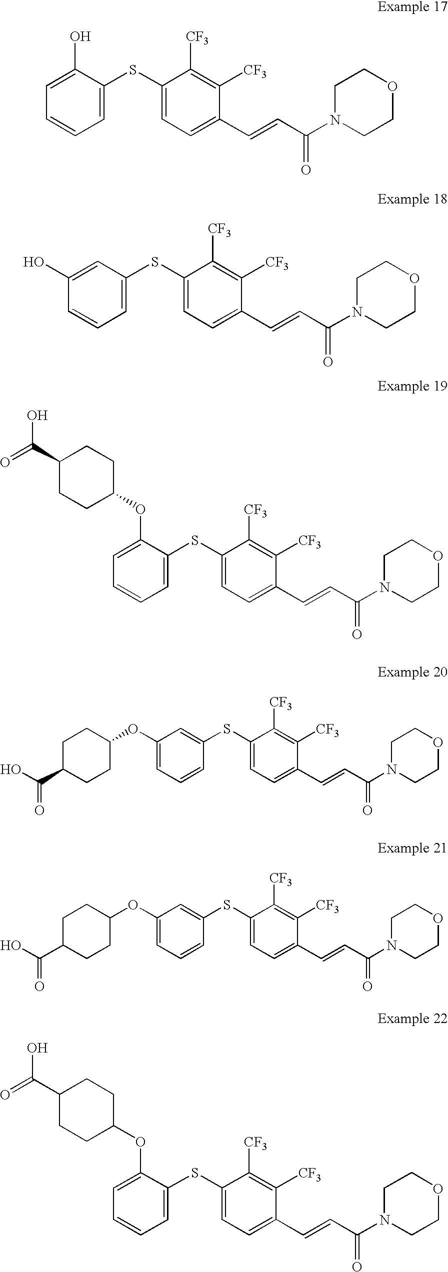

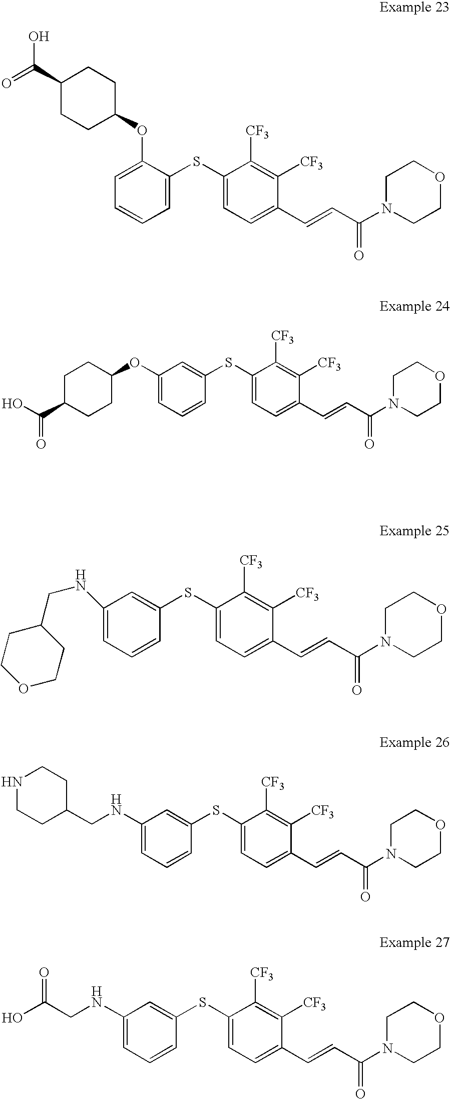

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of formula I: 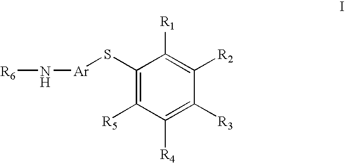
and pharmaceutically-acceptable salts and prodrugs thereof,
wherein R₁, R₂, R₃, R₄, and R₅ are each independently selected from hydrogen, alkyl, alkenyl, alkenoxy, alkynyl,aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups,
R₆ is selected from unsubstituted alkyls, unsubstituted saturated cycloalkyls, unsubstituted carboxyalkyls, and unsubstituted heterocyclylalkyls,
wherein the unsubstituted saturated cycloalkyls, unsubstituted carboxyalkyls, and unsubstituted heterocyclylalkyls are bonded to the NH of formula I through the alkyl group,
wherein the unsubstituted carboxyalkyls comprise a branched alkyl chain,
with the proviso that the heterocyclylalkyl is not 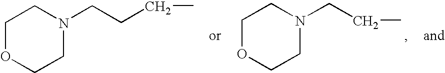
with the proviso that at least one of R₁ and R₃ is selected from:
A. cinnamides selected from cis-cinnamide or trans-cinnamide defined as 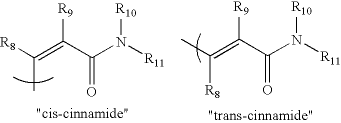
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, sulfonate, sulfonyl, thio, and other carbonyl-containing groups;
B. substituents of formula IV: 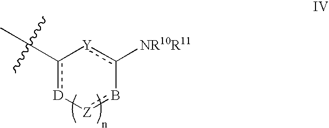
wherein D, B, Y and Z are each independently selected from the group consisting of —CR³¹═, —CR³²R³³—, —C(O)—, —O—, —SO₂—, —S—, —N═, and —NR³⁴—;
n is an integer of zero to three; and
R³¹, R³², R³³ and R³⁴ are each independently selected from the group consisting of hydrogen, alkyl, carboxy, hydroxyalkyl, monoalkylaminocarbonylalkyl, dialkylaminocarbonylalkyl and carboxyalkyl;
C. cyclopropyl derivatives selected from cis-cyclopropanoic acid, trans-cyclopropanoic acid, cis-cyclopropanamide and trans-cyclopropanamide defined as 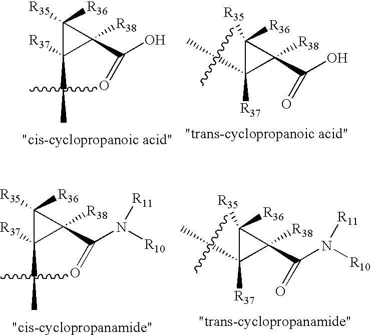
wherein R₃₅ and R₃₆ are each independently selected from the group consisting of hydrogen, alkyl, carboxy, hydroxyalkyl, and carboxyalkyl, and
wherein R₃₇ and R₃₈ are each independently selected from the group consisting of hydrogen, alkyl, carboxyalkyl, monoalkylaminocarbonylalkyl, and dialkylaminocarbonylalkyl;
D. substituents of formula VI: 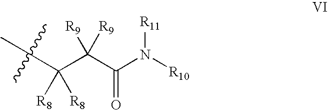
wherein R₈ and R₉ are as defined above;
E. cinnamic acids of formula VII: 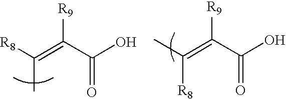
“cis-cinnamic acid” “trans-cinnamic acid”
wherein R₈ and R₉ are as defined above;
wherein:
R₁₀ and R₁₁ are each independently selected from hydrogen, alkanoyl, alkyl, alkenyl, alkynyl, alkoxy, amido, aryl, arylalkyl, carboxy, cyano, cycloalkyl, ester, ether, heterocyclyl, hydroxy, ketone, nitro, sulfonyl thio, and other carbonyl-containing groups, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and
wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, and cyclopropyl derivatives as defined above, and R₂ and R₃, R₃ and R₄, and R₄ and R₅ can be joined to form a 5- to 7- membered cycloalkyl, aryl or heterocyclyl ring when R₁ is selected from cinnamides, substituents of formula IV, substituents of formula VI, and cyclopropyl derivatives as defined above,
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups.

2. The compound according to claim 1, wherein R₆ is selected from C₁₋₆ unsubstituted alkyls.

3. The compound according to claim 2, wherein R₆ is methyl.

4. The compound according to claim 1, wherein R₆ is selected from C₂₋₇ unsubstituted carboxyalkyls.

5. The compound according to claim 4 wherein R₆ is —CH(CH₃)—CH₂—CH₂—C(O)—OH.

6. The compound according to claim 1, wherein R₆ is selected from C₃₋₈ unsubstituted saturated cycloalkyls.

7. The compound according to claim 6, wherein R₆ is selected from cyclobutyl, cyclopentyl, cyclohexyl, cycloheptyl, and bicyclo[2.2.1 ]heptyl, and cyclooctyl.

8. The compound according to claim 1, wherein R₆ is selected from unsubstituted heterocyclylalkyls.

9. The compound according to claim 8, wherein R₆ is selected from imidazolyl(C₁-C₆)alkyl, tetrahydropyranyl(C₁-C₆)alkyl, piperidinyl(C₁-C₆)alkyl and pyridyl(C₁-C₆)alkyl.

10. The compound according to claim 8, wherein the unsubstituted heterocyclylalkyl comprises a heterocycle selected from acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydropyranyl, tetrahydroisoquinolyl, tetrahydroquinolyl, tetrazolyl, thiadiazolyl, thiazolidinyl, thiazolyl, thienyl, thiomorpholinyl, triazolyl, bridged bicyclic groups wherein a monocyclic heterocyclic group is bridged by an alkylene group,
and compounds of the formula 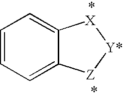
where X* and Z* are independently selected from —CH₂—, —CH₂NH—, —CH₂O—, —NH— and —O—, with the proviso that at least one of X* and Z* is not —CH₂—, and Y* is selected from —C(O)— and —(C(R″)₂)ᵥ—, where R″ is hydrogen or alkyl of one to four carbons, and v is 1-3.

11. A compound of formula III: 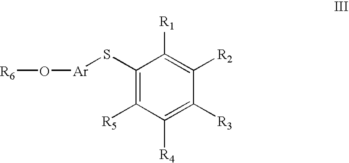
and pharmaceutically-acceptable salts and prodrugs thereof,
wherein R₁, R₂, R₃, R₄, and R₅ are each independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, perfluoroalkyl, sulfonyl, sulfonate, thio, and other carbonyl-containing groups,
wherein R₁ and R₂, and R₄ and R₅ can be joined to form a 5- to 7-membered cycloalkyl, aryl or heterocyclyl ring when R₃ is selected from cinnamides, substituents of formula IV, substituents of formula VI, and cyclopropyl derivatives as defined above, and R₂ and R₃, R₃ and R₄, and R₄ and R₅ can be joined to form a 5- to 7-membered cycloalkyl, aryl or heterocyclyl ring when R, is selected from cinnamides, substituents of formula IV, substituents of formula VI, and cyclopropyl derivatives as defined above,
wherein R₆ is carboxycycloalkyl,
with the proviso that at least one of R₁ and R₃ is selected from:
A. cinnamides selected from cis-cinnamide or trans-cinnamide defined as 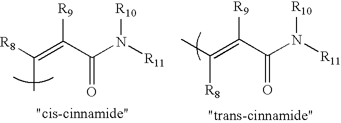
wherein R₈ and R₉ are each independently selected from hydrogen, aldehyde, alkyl, alkenyl, alkynyl, alkoxy, amido, amino, aryl, carboxy, cyano, cycloalkyl, ester, ether, halogen, hydroxy, ketone, nitro, and other carbonyl-containing groups;
B. substituents of formula IV: 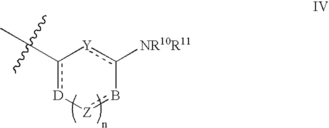
wherein D, B, Y and Z are each independently selected from the group consisting of —CR³¹═, —CR³²R³³—, —C(O)—, —O—, —SO₂—, —S—, —N═, and —NR³—;
n is an integer of zero to three; and
R³¹, R³², R³³ and R³⁴ are each independently selected from the group consisting of hydrogen, alkyl, carboxy, hydroxyalkyl, monoalkylaminocarbonyl alkyl, dialkylaminocarbonylalkyl and carboxyalkyl;
C. cyclopropyl derivatives selected from cis-cyclopropanoic acid, trans-cyclopropanoic acid, cis-cyclopropanamide and trans-cyclopropanamide defined as 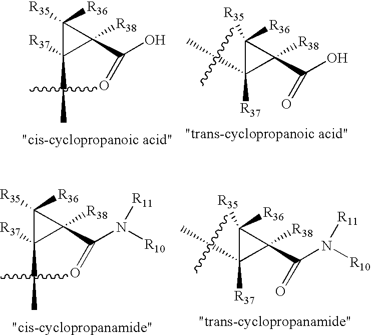
wherein R₃₅ and R₃₆ are each independently selected from the group consisting of hydrogen, alkyl, carboxy, hydroxyalkyl, and carboxyalkyl, and
wherein R₃₇ and R₃₈ are each independently selected from the group consisting of hydrogen, alkyl, carboxyalkyl, monoalkylaminocarbonylalkyl, and dialkylaminocarbonylalkyl;
D. substituents of formula VI: 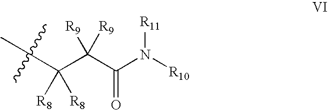
wherein R₈ and R₉ are as defined above; and
E. cinnamic acids of formula VII: 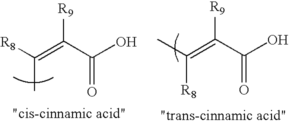
wherein R₈ and R₉ are as defined above;
wherein:
R₁₀ and R₁₁ are each independently selected from hydrogen, alkanoyl, alkyl, alkenyl, alkynyl, alkoxy, amido, aryl, arylalkyl, carboxy, cyano, cycloalkyl, ester, ether, heterocyclyl, hydroxy, ketone, nitro, and other carbonyl-containing groups, or
R₁₀ and R₁₁ are taken together with N to form a heterocyclyl group bonded to at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups, and
wherein Ar is selected from aryl and heteroaryl having at least one substituent independently selected from hydrogen, alkyl, alkenyl, alkenoxy, alkynyl, aldehyde, alkanoyl, alkoxy, amido, amino, aryl, aryloxy, carboxy, cyano, cycloalkyl, ether, ester, halogen, heterocyclyl, hydroxy, ketone, nitro, oxo, perfluoroalkyl, sulfonyl, sulfonate, thio, and other carbonyl-containing groups.

12. The compound according to claim 11, wherein the carboxycycloalkyl of R₆ comprises a cycloalkyl group selected from cyclobutyl, cyclopentyl, cyclohexyl, cycloheptyl, and cyclooctyl.

13. The compound according to claim 12, wherein R₆ is carboxycyclohexyl.

14. The compound according to claim 1, wherein R₁ and R₂ are selected from hydrogen, alkyl, halogen, haloalkyl, and nitro.

15. The compound according to claim 1, wherein R₃ is a “cis-cinnamide” or “trans-cinnamide” and R. is not a “cis-cinnamide” or “trans-cinnamide.”

16. The compound according to claim 1, wherein R₃ is a substituent of formula IV and R₁ is not a substituent of formula IV.

17. The compound according to claim 1, wherein R₃ is a cyclopropyl derivative and R₁ is not a cyclopropyl derivative.

18. The compound according to claim 1, wherein R₃ is a substituent of formula VI and R₁ is not a substituent of formula VI.

19. The compound according to claim 1, wherein R₃ is a substituent of formula VII and R₁ is not a substituent of formula

20. The compound according to claim 1, wherein the compound exhibits an IC₅₀ of less than or equal to about 1.0 μM as determined by an ICAM-1/LFA-1 biochemical interaction assay.

21-23. (canceled)

24. The compound according to claim 1, wherein the compound exhibits an EC₈₀ of less than or equal to about 3.0 μM as determined by a T cell proliferation assay.

25-26. (canceled)

27. A pharmaceutical composition comprising the compound according to claim 1.

28. (canceled)

29. A method of treating an inflammatory disease or inhibiting inflammation, comprising administering to a subject a pharmaceutical composition comprising the compound according to claim 1.

30. A method of treating an immune disease or suppressing an immune response, comprising administering to a subject a pharmaceutical composition comprising the compound according to claim 1.

31-32. (canceled)

33. A method of treating a disease associated with an interaction between ICAM-1 and LFA-1, comprising administering to a subject a pharmaceutical composition comprising the compound according to claim 1.

34-36. (canceled)

37. A method of treating psoriasis, comprising administering to a subject a pharmaceutical composition comprising the compound according to claim 1.

38-41. (canceled)

42. A method for treating a disease or disorder in a mammal, comprising administering to said mammal a therapeutic amount of a compound according to claim 1 or claim 11, wherein the disease or disorder benefits from inhibiting the interaction of LFA-1 with ICAM-1 or ICAM-3, and wherein administering to said mammal inhibits inflammation.

43. A method of inhibiting the interaction of LFA-1 with ICAM-1 or ICAM-3, comprising administering to a mammal an effective amount of a compound according to claim 1 or claim 11, wherein administering to said mammal inhibits inflammation.

44. A method for treating a disease or disorder selected from prophylaxis, reperfusion injury, ischemic-reperfusion injury, pulminary reperfusion injury, stroke, asthma, myocardial infarction, psoriasis, atherosclerosis, atopic dermatitis, hepatitis, adult respiratory distress syndrome, chronic ulceration, lung fibrosis, graft-versus-host disease, chronic obstructive pulmonary disease, Sjögren's syndrome, multiple sclerosis, autoimmune thyroiditis, Graves' disease, glomerulonephritis, systemic lupus erythematosus, diabetes, autoimmune diabetes, primary biliary cirrhosis, autoimmune uveoretinitis, scleroderma, arthritis, Lyme arthritis, fulminant hepatitis, inflammatory liver injury, thyroid diseases, transplant rejection, inflammatory lung injury, radiation pneumonitis, inflammatory bowel diseases, inflammatory glomerular injury, radiation-induced enteritis, peripheral artery occlusion, graft rejection, and cancer, comprising administering to a mammal a therapeutic amount of a compound according to claim 1 or claim 11.
